(19) United States
(12) Patent Application Publication
(10) Pub. No.: US 2026/0207441 A1
(43) Pub. Date: Jul. 23, 2026

(54) COMPOSITION COMPRISING HYALURONIC ACID-BASED POLYION COMPLEX AND HYDROPHILIC OR LIPOPHILIC ANTIOXIDANT

(71) Applicant: L'Oreal, Paris (FR)

(72) Inventors: Yuichi Ikeda, Kawasaki-shi (JP); Shinsuke Okuda, Kawasaki-shi (JP); Mitsuru Shimatani, Kawasaki-shi (JP)

(73) Assignee: L'Oreal, Paris (FR)

(21) Appl. No.: 19/140,234

(22) PCT Filed: Dec. 5, 2023
(86) PCT No.: PCT/JP2023/044120

§ 371 Date: Jun. 17, 2025

(30) Foreign Application Priority Data
Dec. 20, 2022 (JP) 2022-203614
Dec. 20, 2022 (JP) 2022-203615
Jan. 27, 2023 (FR) 2300762
Jan. 30, 2023 (FR) 2300820

(51) Int. Cl. A61K 8/02; A61K 8/41; A61K 8/55; A61K 8/65; A61K 8/67; A61K 8/73; A61K 8/88; A61Q 19/00

(52) U.S. Cl. CPC A61K 8/0216; A61K 8/416; A61K 8/55; A61K 8/65; A61K 8/676; A61K 8/731; A61K 8/735; A61K 8/88; A61Q 19/00

(57) ABSTRACT

The present invention relates to a composition comprising: (a) at least one complex comprising at least one cationic polymer and at least one anionic polymer, at least one cationic polymer and at least one amphoteric polymer, at least one anionic polymer and at least one amphoteric polymer, or at least one amphoteric polymer, and at least one non-polymeric acid having two or more pKa values or salt(s) thereof or at least one non-polymeric base having two or more pKb values or salt(s) thereof; (b) at least one hydrophilic or lipophilic antioxidant; and (c) water, wherein the anionic polymer is selected from hyaluronic acid, salts thereof, and derivatives thereof, and the amphoteric polymer is selected from cationized hyaluronic acid and salts thereof. The composition according to the present invention can be transparent or translucent, or less sticky, and can provide long lasting antioxidative effects.

### TECHNICAL FIELD

The present invention relates to a composition including a polyion complex and a film of a polyion complex, as well as a process for preparing a film by using a polyion complex and a cosmetic process using a polyion complex.

### BACKGROUND ART

Hyaluronic acid is a predominant glucosaminoglycan found in the skin. Thus, the fibroblasts synthesize predominantly collagens, matrix glycoproteins other than collagens (fibronectin, laminin), proteoglycans and elastin. The keratinocytes, for their part, synthesize predominantly sulfated glycosaminoglycans and hyaluronic acid. Hyaluronic acid is also called hyaluronan (HA).

Hyaluronic acid is present in the free state in the epidermis and in the dermis and is responsible for turgescence of the skin. This polysaccharide can in fact retain a large volume of water, corresponding to up to 1000 times its weight. In this sense, hyaluronic acid plays an important role in increasing the amounts of water bound in the tissue, and also in the mechanical properties of the skin and in wrinkle formation.

Hyaluronic acid has been widely used as a cosmetic ingredient due to its high moisturizing effects.

However, an aqueous solution of hyaluronic acid is sticky, and this may result in an uncomfortable texture. Also, a hyaluronic acid film, which is formed when an aqueous solution of hyaluronic acid dries on the skin, is sticky, and such a film may also result in an uncomfortable texture.

WO 2021/125069 discloses reducing stickiness due to hyaluronic acid by forming a polyion complex particle with a cationic polymer and hyaluronic acid as an anionic polymer.

### DISCLOSURE OF INVENTION

There has been a need for a composition including a hyaluronic acid-based polyion complex, which can be transparent or translucent and can provide long lasting antioxidative effects.

There also has been a need for a composition including a hyaluronic acid-based polyion complex, which can be less sticky and can provide long lasting antioxidative effects.

Thus, a first objective of the present invention is to provide a composition which can be transparent or translucent, or less sticky, while the composition can provide long lasting antioxidative effects.

The above objective of the present invention can be achieved by a composition, comprising:
(a) at least one polyion complex, comprising
   at least one cationic polymer and at least one anionic polymer,
   at least one cationic polymer and at least one amphoteric polymer,
   at least one anionic polymer and at least one amphoteric polymer, or
   at least one amphoteric polymer,
   and
   at least one non-polymeric acid having two or more pKa values or salt(s) thereof, or
   at least one non-polymeric base having two or more pKb values or salt(s) thereof;
(b) at least one hydrophilic or lipophilic antioxidant; and
(c) water,
wherein
the anionic polymer is selected from hyaluronic acid, salts thereof, and derivatives thereof,
and
the amphoteric polymer is selected from cationized hyaluronic acid and salts thereof.

The ingredient (b) in the composition according to the present invention may be at least one hydrophilic antioxidant or at least one lipophilic antioxidant.

The cationized hyaluronic acid may have at least one quaternary ammonium group-containing group and has a degree of cationization of 0.05 to 0.6, preferably from 0.1 to 0.5, and more preferably from 0.15 to 0.4.

The cationic polymer may have at least one positively chargeable and/or positively charged moiety selected from the group consisting of a primary, secondary or tertiary amino group, a quaternary ammonium group, a guanidine group, a biguanide group, an imidazole group, an imino group, and a pyridyl group.

The cationic polymer may be selected from the group consisting of cyclopolymers of alkyldiallylamine and cyclopolymers of dialkyldiallylammonium such as (co)polydiallyldialkyl ammonium chloride, (co)polyamines such as (co)polylysines, cationic (co)polyaminoacids such as collagen, cationic cellulose polymers, and salts thereof.

The total amount of the cationic and/or anionic and/or amphoteric polymer(s) in the composition according to the present invention may be from 0.01% to 15% by weight, preferably from 0.03% to 10% by weight, and more preferably from 0.05% to 5% by weight, relative to the total weight of the composition.

The non-polymeric acid having two or more pKa values or salt(s) thereof may be an organic acid or salt(s) thereof, and preferably a hydrophilic or water-soluble organic acid or salt(s) thereof, and more preferably phytic acid or salts thereof.

The amount of the non-polymeric acid having two or more pKa values or salt(s) thereof or non-polymeric base having two or more pKb values or salt(s) thereof in the composition according to the present invention may be from 0.001% to 15% by weight, preferably from 0.005% to 10% by weight, and more preferably from 0.01% to 5% by weight, relative to the total weight of the composition.

The (b) hydrophilic antioxidant may be selected from ascorbic acid, derivatives thereof, salts 50 thereof, and mixtures thereof.

The amount of the (b) hydrophilic antioxidant in the composition according to the present invention may be from 0.01% to 30% by weight, preferably from 0.05% to 25% by weight, and more preferably from 0.1% to 20% by weight, relative to the total weight of the composition.

The (b) lipophilic antioxidant may be selected from the group consisting of pentaerythrityl tetra-di-t-butyl hydroxyhydrocinnamate, nordihydroguaiaretic acid, propyl gallate, butylated hydroxytoluene, butylated hydroxyanisole, ascorbyl palmitate, tocopherol and mixtures thereof.

The amount of the (b) lipophilic antioxidant in the composition according to the present invention may be from 0.001% to 15% by weight, preferably from 0.005% to 10% by weight, and more preferably from 0.01% to 5% by weight, relative to the total weight of the composition.

A second objective of the present invention is to provide a process to prepare a film which can provide long lasting antioxidative effects.

The above objective of the present invention can be achieved by a process for preparing a film, preferably a cosmetic film, comprising:
applying onto a substrate, preferably a keratin substance, the composition according to the present invention; and
drying the composition.

A third objective of the present invention is to provide a film which can provide long lasting antioxidative effects.

The above objective of the present invention can be achieved by:
(1) A film, preferably a cosmetic film, prepared by a process comprising:
   applying onto a substrate, preferably a keratin substance, the composition according to the present invention; and
   drying the composition,
or
(2) A film, preferably a cosmetic film, comprising:
   (a) at least one polyion complex, comprising
   at least one cationic polymer and at least one anionic polymer,
   at least one cationic polymer and at least one amphoteric polymer,
   at least one anionic polymer and at least one amphoteric polymer, or
   at least one amphoteric polymer, and
   at least one non-polymeric acid having two or more pKa values or salt(s) thereof, or
   at least one non-polymeric base having two or more pKb values or salt(s) thereof;
   (b) at least one hydrophilic or lipophilic antioxidant;
   and
   optionally (d) at least one surfactant, preferably selected from non-ionic surfactants,
   and more preferably selected from polyglyceryl fatty acid esters,
   wherein
   the anionic polymer is selected from hyaluronic acid, salts thereof, and derivatives thereof; and
   the amphoteric polymer is selected from cationized hyaluronic acid and salts thereof.

The present invention also relates to a cosmetic process for a keratin substance such as skin, comprising
applying to the keratin substance the composition according to the present invention; and drying the composition to form a cosmetic film on the keratin substance.

### BEST MODE FOR CARRYING OUT THE INVENTION

After diligent research, the inventors have discovered that it is possible to provide a composition which can be transparent or translucent, or less sticky, while the composition can provide long lasting antioxidant effects. Thus, the composition according to the present invention comprises:
(a) at least one polyion complex, comprising
   at least one cationic polymer and at least one anionic polymer,
   at least one cationic polymer and at least one amphoteric polymer,
   at least one anionic polymer and at least one amphoteric polymer, or
   at least one amphoteric polymer,
   and
   at least one non-polymeric acid having two or more pKa values or salt(s) thereof, or
   at least one non-polymeric base having two or more pKb values or salt(s) thereof;
(b) at least one hydrophilic or lipophilic antioxidant; and
(c) water,
wherein
the anionic polymer is selected from hyaluronic acid, salts thereof, and derivatives thereof,
and
the amphoteric polymer is selected from cationized hyaluronic acid and salts thereof.

Further, the inventors have discovered that it is possible to provide a process to prepare a film which can provide long lasting antioxidative effects. Thus, the process according to the present invention is a process for preparing a film, preferably a cosmetic film, the process comprising:
applying onto a substrate, preferably a keratin substance, the composition according to the present invention; and
drying the composition.

Furthermore, the inventors have discovered that it is possible to provide a film which can provide long lasting antioxidant effects. Thus, the film according to the present invention is
(1) A film, preferably a cosmetic film, prepared by a process comprising:
   applying onto a substrate, preferably a keratin substance, the composition according to the present invention; and
   drying the composition,
or
(2) A film, preferably a cosmetic film, comprising:
   (a) at least one polyion complex, comprising
   at least one cationic polymer and at least one anionic polymer,
   at least one cationic polymer and at least one amphoteric polymer,
   at least one anionic polymer and at least one amphoteric polymer, or
   at least one amphoteric polymer, and
   at least one non-polymeric acid having two or more pKa values or salt(s) thereof, or
   at least one non-polymeric base having two or more pKb values or salt(s) thereof;
   (b) at least one hydrophilic or lipophilic antioxidant;
   and
   optionally (d) at least one surfactant, preferably selected from non-ionic surfactants,
   and more preferably selected from polyglyceryl fatty acid esters,
   wherein
   the anionic polymer is selected from hyaluronic acid, salts thereof, and derivatives
   thereof; and
   the amphoteric polymer is selected from cationized hyaluronic acid and salts thereof.

The composition and film according to the present invention can be transparent or translucent, if at least one hydrophilic antioxidant is used as the ingredient (b).

On the other hand, if at least one lipophilic antioxidant is used as the ingredient (b), the stickiness of the composition according to the present invention can be reduced as compared to a composition including hyaluronic acid, which does not form a polyion complex.

The composition and film according to the present invention can provide long lasting antioxidative effects.

Thus, the present invention can be useful for protecting skin from damage caused by the peroxidation of, for example, unsaturated lipids such as sebum, wherein the damage may be alteration of desquamation, squalene decrease, acne and black heads, dull skin, and aging, such as the formation of wrinkles and/or fine lines.

Further, the composition and film according to the present invention can provide long lasting cosmetic effects and/or improved moisturizing texture based on hyaluronic acid or cationized hyaluronic acid.

It is possible to easily prepare a film comprising a polyion complex, wherein the film may include at least one surfactant, by applying the composition onto a substrate, preferably a keratin substance such as skin and hair, and more preferably skin, and drying the composition.

The stickiness of the film according to the present invention can be reduced as compared to a film of hyaluronic acid which does not form a polyion complex.

The film according to the present invention may be porous. The surface of the film according to the present invention may not be flat but may be rough.

The film according to the present invention can have a variety of cosmetic functions. For example, the film can provide moisturizing effects based on hyaluronic acid or cationized hyaluronic acid in the polyion complex.

The film according to the present invention is capable of capturing sebum, matting the appearance of a keratin substance such as skin, absorbing or adsorbing malodour and/or protecting the keratin substance from, for example, dirt or pollutants.

If the film includes at least one cosmetic active ingredient such as oil(s), the film can also have cosmetic effects provided by the cosmetic active ingredient(s). For example, if the polyion complex film includes at least one cosmetic active ingredient selected from UV filters, anti-aging agents other than the (b) hydrophilic or lipophilic antioxidant, anti-sebum agents, deodorant agents, anti-perspirant agents, whitening agents and a mixture thereof, the film can filter UV rays, treat the aging of the skin, absorb sebum on the skin, control odors on the skin, control the perspiration on the skin, and/or whiten the skin.

The film according to the present invention can be transparent or translucent, and therefore, may not be easy to perceive, even when the film is relatively thick.

Further, the film according to the present invention is water-resistant, and therefore, it can remain on a keratin substance such as skin even if the surface of the keratin substance is wet due to, for example, sweat or rain.

Furthermore, the film according to the present invention can be easily removed from a keratin substance such as skin under alkaline conditions. Therefore, the film according to the present invention is difficult to remove with water, while it can be easily removed with a soap which can provide alkaline conditions.

Thus, if the film according to the present invention includes a hydrophilic or water-soluble UV filter, the film according to the present invention can exhibit UV shielding effects which are resistant to water (water-proof) and can be long-lasting, but can be easily removed with a soap which can provide alkaline conditions.

Hereinafter, the composition, process, film and the like according to the present invention will be explained in a more detailed manner.

### [Polyion Complex]

The composition according to the present invention includes (a) at least one polyion complex.

Two or more different types of (a) polyion complexes may be used in combination. Thus, a single type of (a) polyion complex or a combination of different types of (a) polyion complexes may be used.

The size of the (a) polyion complex may be from 5 nm to 100 μm, preferably from 100 nm to 50 μm, more preferably from 200 nm to 40 μm, and even more preferably from 500 nm to 30 μm. The size less than 1 μm can be measured by a dynamic light scattering method, and the size more than 1 μm can be measured by an optical microscope. This size may be based on a number average diameter.

The amount of the (a) polyion complex(es) in the composition according to the present invention may be 0.01% by weight or more, preferably 0.05% by weight or more, and more preferably 0.1% by weight or more, relative to the total weight of the composition.

The amount of the (a) polyion complex(es) in the composition according to the present invention may be 15% by weight or less, preferably 10% by weight or less, and more preferably 5% by weight or less, relative to the total weight of the composition.

The amount of the (a) polyion complex(es) in the composition according to the present invention may be from 0.01% to 15% by weight, preferably from 0.05% to 10% by weight, and more preferably from 0.1% to 5% by weight, relative to the total weight of the composition.

The (a) polyion complex includes at least one polymer or a combination of polymers.

Specifically, the (a) polyion complex includes:
(1) at least one cationic polymer and at least one anionic polymer;
(2) at least one cationic polymer and at least one amphoteric polymer;
(3) at least one anionic polymer and at least one amphoteric polymer; or
(4) at least one amphoteric polymer.

There is no limit to the type of the cationic polymers. Two or more different types of cationic polymers may be used in combination. Thus, a single type of cationic polymer or a combination of different types of cationic polymers may be used.

In the above (1), the ratio of the amount, for example the chemical equivalent, of the cationic polymer(s)/the anionic polymer(s) may be 0.05-18, preferably 0.1-10, and more preferably 0.5-5.0. In particular, it may be preferable that the number of the cationic groups of the cationic polymer(s)/the number of anionic groups of the anionic polymer(s) be 0.05-18, more preferably 0.1-10, and even more preferably 0.5-5.0.

In the above (2), the ratio of the amount, for example the chemical equivalent, of the cationic polymer(s)/the amphoteric polymer(s) may be 0.05-18, preferably 0.1-10, and more preferably 0.5-5.0. In particular, it may be preferable that the number of the cationic groups of the cationic polymer(s)/the number of cationic and anionic groups of the amphoteric polymer(s) be 0.05-18, more preferably 0.1-10, and even more preferably 0.5-5.0.

In the above (3), the ratio of the amount, for example the chemical equivalent, of the anionic polymer(s)/the amphoteric polymer(s) may be 0.05-18, preferably 0.1-10, and more preferably 0.5-5.0. In particular, it may be preferable that the number of the anionic groups of the anionic polymer(s)/the number of cationic and anionic groups of the amphoteric polymer(s) be 0.05-18, more preferably 0.1-10, and even more preferably 0.5-5.0.

The total amount of the polymer(s) according to any one of the above (1) to (4) in the composition according to the present invention may be 0.01% by weight or more, preferably 0.03% by weight or more, and more preferably 0.05% by weight or more, relative to the total weight of the composition.

The total amount of the polymer(s) according to any one of the above (1) to (4) in the composition according to the present invention may be 15% by weight or less, preferably 10% by weight or less, and more preferably 5% by weight or less, relative to the total weight of the composition.

The total amount of the polymer(s) according to any one of the above (1) to (4) in the composition according to the present invention may be from 0.01% to 15% by weight, preferably from 0.03% to 10% by weight, and more preferably from 0.05% to 5% by weight, relative to the total weight of the composition.

### (Cationic Polymer)

A cationic polymer has a positive charge density. The charge density of the cationic polymer may be from 0.01 meq/g to 20 meq/g, preferably from 0.05 to 15 meq/g, and more preferably from 0.1 to 10 meq/g.

It may be preferable that the molecular weight of the cationic polymer be 500 or more, preferably 1000 or more, more preferably 2000 or more, and even more preferably 3000 or more.

Unless otherwise defined in the descriptions, “molecular weight” means a number average molecular weight.

The cationic polymer may have at least one positively chargeable and/or positively charged moiety selected from the group consisting of a primary, secondary or tertiary amino group, a quaternary ammonium group, a guanidine group, a biguanide group, an imidazole group, an imino group, and a pyridyl group. The term (primary) “amino group” here means a group of —NH₂.

The cationic polymer may be a homopolymer or a copolymer. The term “copolymer” is understood to mean both copolymers obtained from two kinds of monomers and those obtained from more than two kinds of monomers, such as terpolymers obtained from three kinds of monomers.

The cationic polymer may be selected from natural and synthetic cationic polymers. Non-limiting examples of the cationic polymers are as follows.

(1) Homopolymers and copolymers derived from acrylic or methacrylic esters and amides and comprising at least one unit chosen from units of the following formulas:

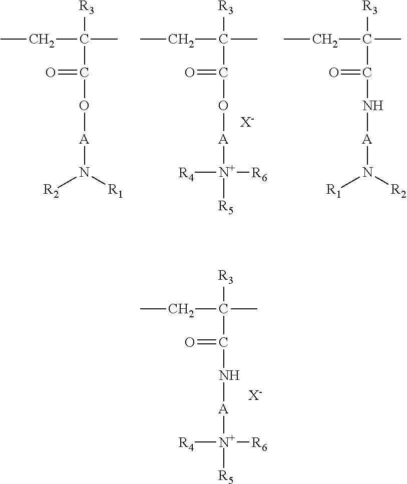

wherein:
R₁ and R₂, which may be identical or different, are chosen from hydrogen and alkyl groups comprising from 1 to 6 carbon atoms, for instance, methyl and ethyl groups;
R₃, which may be identical or different, is chosen from hydrogen and CH₃;
the symbols A, which may be identical or different, are chosen from linear or branched alkyl groups comprising from 1 to 6 carbon atoms, for example, from 2 to 3 carbon atoms and hydroxyalkyl groups comprising from 1 to 4 carbon atoms;
R₄, R₅, and R₆, which may be identical or different, are chosen from alkyl groups comprising from 1 to 18 carbon atoms and benzyl groups, and in at least one embodiment, alkyl groups comprising from 1 to 6 carbon atoms; and
X is an anion derived from an inorganic or organic acid, such as methosulphate anions and halides, for instance chloride and bromide.

The copolymers of family (1) may also comprise at least one unit derived from comonomers which may be chosen from acrylamides, methacrylamides, diacetone acrylamides, acrylamides and methacrylamides substituted on the nitrogen atom with (C₁-C₄) lower alkyl groups, groups derived from acrylic or methacrylic acids and esters thereof, vinyllactams such as vinylpyrrolidone and vinylcaprolactam, and vinyl esters.

Examples of copolymers of family (1) include, but are not limited to:
copolymers of acrylamide and of dimethylaminoethyl methacrylate quaternized with dimethyl sulphate or with a dimethyl halide,
copolymers of acrylamide and of methacryloyloxyethyltrimethylammonium chloride described, for example, in European Patent Application No. 0 080 976,
copolymers of acrylamide and of methacryloyloxyethyltrimethylammonium methosulphate, quaternized or nonquaternized vinylpyrrolidone/dialkylaminoalkyl acrylate or methacrylate copolymers, described, for example, in French Patent Nos. 2 077 143 and 2 393 573, dimethylaminoethyl methacrylate/vinylcaprolactam/vinylpyrrolidone terpolymers, vinylpyrrolidone/methacrylamidopropyldimethylamine copolymers, quaternized vinylpyrrolidone/dimethylaminopropylmethacrylamide copolymers, and crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with a compound containing an olefinic unsaturation, for example, methylenebisacrylamide.

(2) Cationic cellulose polymers such as cellulose ether derivatives comprising one or more quaternary ammonium groups described, for example, in French Patent No. 1 492 597, such as the polymers sold under the names “JR” (JR 400, JR 125, JR 30M) or “LR” (LR 400, LR 30M) by the company Union Carbide Corporation. These polymers are also defined in the CTFA dictionary as quaternary ammoniums of hydroxyethylcellulose that have reacted with an epoxide substituted with a trimethylammonium group.

It is preferable that the cationic cellulose polymer have at least one quaternary ammonium group, preferably a quaternary trialkyl ammonium group, and more preferably a quaternary trimethyl ammonium group.

The quaternary ammonium group may be present in a quaternary ammonium group-containing group which may be represented by the following chemical formula (I):

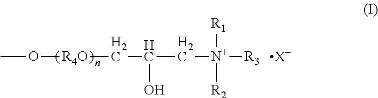

wherein
each of R₁ and R₂ denotes a C₁₋₃ alkyl group, preferably a methyl or ethyl group, and more preferably a methyl group,
R₃ denotes a C₁₋₂₄ alkyl group, preferably a methyl or ethyl group, and more preferably methyl group,
X— denotes an anion, preferably a halide, and more preferably a chloride,
n denotes an integer from 0-30, preferably 0-10, and more preferably 0, and
R₄ denotes a C₁₋₄ alkylene group, preferably an ethylene or propylene group.

The leftmost ether bond (—O—) in the above chemical formula (I) can attach to the sugar ring of the polysaccharide.

It is preferable that the quaternary ammonium group-containing group be —O—CH₂—CH(OH)—CH₂—N⁺(CH₃)₃.

(3) Cationic cellulose polymers such as cellulose copolymers and cellulose derivatives grafted with a water-soluble monomer of quaternary ammonium, and described, for example, in U.S. Pat. No. 4,131,576, such as hydroxyalkylcelluloses, for instance, hydroxymethyl-, hydroxyethyl-, and hydroxypropylcelluloses grafted, for example, with a salt chosen from methacryloylethyltrimethylammonium, methacrylamidopropyltrimethylammonium, and dimethyldiallylammonium salts.

Commercial products corresponding to these polymers include, for example, the products sold under the name “Celquat® L 200” and “Celquat® H 100” by the company National Starch.

(4) Non-cellulose-based cationic polysaccharides described in U.S. Pat. Nos. 3,589,578 and 4,031,307, such as guar gums comprising cationic trialkylammonium groups, cationic hyaluronic acid, and dextran hydroxypropyl trimonium chloride. Guar gums modified with a salt, for example the chloride, of 2,3-epoxypropyltrimethylammonium (guar hydroxypropyltrimonium chloride) may also be used.

Such products are sold, for instance, under the trade names JAGUAR® C13 S, JAGUAR® C15, JAGUAR® C17, and JAGUAR® C162 by the company MEYHALL.

(5) Polymers comprising piperazinyl units and divalent alkylene or hydroxyalkylene groups comprising straight or branched chains, optionally interrupted with at least one entity chosen from oxygen, sulphur, nitrogen, aromatic rings, and heterocyclic rings, and also the oxidation and/or quaternization products of these polymers. Such polymers are described, for example, in French Patent Nos. 2 162 025 and 2 280 361.

(6) Water-soluble polyamino amides prepared, for example, by polycondensation of an acidic compound with a polyamine; these polyamino amides possibly being crosslinked with an entity chosen from epihalohydrins; diepoxides; dianhydrides; unsaturated dianhydrides; bisunsaturated derivatives; bishalohydrins; bisazetidiniums; bishaloacyidiamines; bisalkyl halides; oligomers resulting from the reaction of a difunctional compound which is reactive with an entity chosen from bishalohydrins, bisazetidiniums, bishaloacyldiamines, bisalkyl halides, epihalohydrins, diepoxides, and bisunsaturated derivatives; the crosslinking agent being used in an amount ranging from 0.025 to 0.35 mol per amine group of the polyamino amide; these polyamino amides optionally being alkylated or, if they comprise at least one tertiary amine function, they may be quaternized. Such polymers are described, for example, in French Patent Nos. 2 252 840 and 2 368 508.

(7) Polyamino amide derivatives resulting from the condensation of polyalkylene polyamines with polycarboxylic acids, followed by alkylation with difunctional agents, for example, adipic acid/dialkylaminohydroxyalkyldialkylenetriamine polymers in which the alkyl group comprises from 1 to 4 carbon atoms, such as methyl, ethyl, and propyl groups, and the alkylene group comprises from 1 to 4 carbon atoms, such as an ethylene group. Such polymers are described, for instance, in French Patent No. 1 583 363. In at least one embodiment, these derivatives may be chosen from adipic acid/dimethylaminohydroxypropyldiethylenetriamine polymers.

(8) Polymers obtained by reaction of a polyalkylene polyamine comprising two primary amine groups and at least one secondary amine group, with a dicarboxylic acid chosen from diglycolic acid and saturated aliphatic dicarboxylic acids comprising from 3 to 8 carbon atoms. The molar ratio of the polyalkylene polyamine to the dicarboxylic acid may range from 0.8:1 to 1.4:1; the polyamino amide resulting therefrom being reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

(9) Cyclopolymers of alkyldiallylamine and cyclopolymers of dialkyldiallyl-ammonium, such as homopolymers and copolymers comprising, as the main constituent of the chain, at least one unit chosen from units of formulas (Ia) and (Ib):

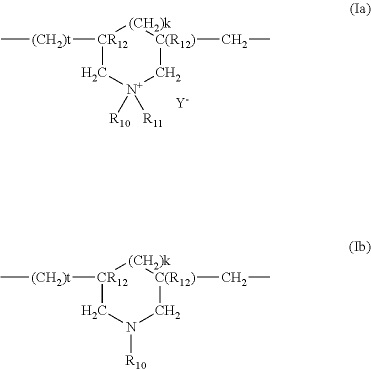

wherein:
k and t, which may be identical or different, are equal to 0 or 1, the sum k+t being equal to 1;
R₁₂ is chosen from hydrogen and methyl groups; R₁₀ and R₁₁, which may be identical or different, are chosen from alkyl groups comprising from 1 to 6 carbon atoms, hydroxyalkyl groups in which the alkyl group comprises, for example, from 1 to 5 carbon atoms, and lower (C₁-C₄)amidoalkyl groups, or R₁₀ and Ru may form, together with the nitrogen atom to which they are attached, heterocyclic groups such as piperidinyl and morpholinyl; and
Y′ is an anion such as bromide, chloride, acetate, borate, citrate, tartrate, bisulphate, bisulphite, sulphate, and phosphate. These polymers are described, for example, in French Patent No. 2 080 759 and in its Certificate of Addition 2 190 406.

In one embodiment, R₁₀ and R₁₁, which may be identical or different, are chosen from alkyl groups comprising from 1 to 4 carbon atoms.

Examples of such polymers include, but are not limited to, (co)polydiallyldialkyl ammonium chloride such as the dimethyidiallylammonium chloride homopolymer sold under the name “MERQUAT® 100” by the company CALGON (and its homologues of low weight-average molecular mass) and the copolymers of diallyldimethylammonium chloride and of acrylamide sold under the name “MERQUAT® 550”.

(10) Quaternary diammonium polymers comprising at least one repeating unit of formula (II):

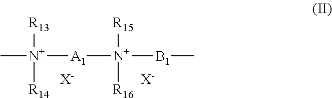

wherein:
R₁₃, R₁₄, R₁₅, and R₁₆, which may be identical or different, are chosen from aliphatic, alicyclic, and arylaliphatic groups comprising from 1 to 20 carbon atoms and lower hydroxyalkyl aliphatic groups, or alternatively R₁₃, R₁₄, R₁₅, and R₁₆ may form, together or separately, with the nitrogen atoms to which they are attached, heterocycles optionally comprising a second heteroatom other than nitrogen, or alternatively R₁₃, R₁₄, R₁₅, and R₁₆, which may be identical or different, are chosen from linear or branched C₁-C₆ alkyl groups substituted with at least one group chosen from nitrile groups, ester groups, acyl groups, amide groups, —CO—O—R₁₇-E groups, and —CO—NH—R₁₇-E groups, wherein R₁₇ is an alkylene group and E is a quaternary ammonium group;
A₁ and B₁, which may be identical or different, are chosen from polymethylene groups comprising from 2 to 20 carbon atoms, which may be linear or branched, saturated or unsaturated, and which may comprise, linked or intercalated in the main chain, at least one entity chosen from aromatic rings, oxygen, sulphur, sulphoxide groups, sulphone groups, disulphide groups, amino groups, alkylamino groups, hydroxyl groups, quaternary ammonium groups, ureido groups, amide groups, and ester groups, and
X⁻ is an anion derived from an inorganic or organic acid;
A₁, R₁₃, and R₁₅ may form, together with the two nitrogen atoms to which they are attached, a piperazine ring;
if A₁ is chosen from linear or branched, saturated or unsaturated alkylene or hydroxyalkylene groups, B₁ may be chosen from:

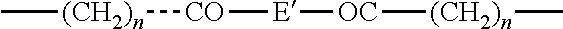

wherein E′ is chosen from:
a) glycol residues of formula —O—Z—O—, wherein Z is chosen from linear or branched hydrocarbon-based groups and groups of the following formulas:

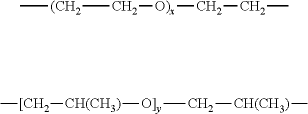

wherein x and y, which may be identical or different, are chosen from integers ranging from 1 to 4, which represent a defined and unique degree of polymerization, and numbers ranging from 1 to 4, which represent an average degree of polymerization;
b) bis-secondary diamine residue such as piperazine derivatives;
c) bis-primary diamine residues of formula —NH—Y—NH—, wherein Y is chosen from linear or branched hydrocarbon-based groups and the divalent group —CH₂—CH₂—S—S—CH₂—CH₂—; and
d) ureylene groups of formula —NH—CO—NH—.

In at least one embodiment, X⁻ is an anion such as chloride or bromide.

Polymers of this type are described, for example, in French Patent Nos. 2 320 330; 2 270 846; 2 316 271; 2 336 434; and 2 413 907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020.

Non-limiting examples of such polymers include those comprising at least one repeating unit of formula (III):

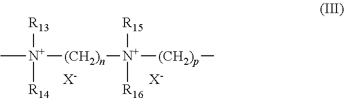

wherein
R₁₃, R₁₄, R₁₅, and R₁₆, which may be identical or different, are chosen from alkyl and hydroxyalkyl groups comprising from 1 to 4 carbon atoms, n and p, which may be identical or different, are integers ranging from 2 to 20, and X⁻ is an anion derived from an inorganic or organic acid.

(11) Polyquaternary ammonium polymers comprising units of formula (IV):

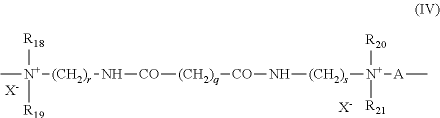

wherein:
R₁₈, R₁₉, R₂₀, and R₂₁, which may be identical or different, are chosen from hydrogen, methyl groups, ethyl groups, propyl groups, β-hydroxyethyl groups, β-hydroxypropyl groups, —CH₂CH₂(OCH₂CH₂)ₚOH groups, wherein p is chosen from integers ranging from 0 to 6, with the proviso that R₁₈, R₁₉, R₂₀, and R₂₁ are not simultaneously hydrogen,
r and s, which may be identical or different, are chosen from integers ranging from 1 to 6,
q is chosen from integers ranging from 0 to 34,
X⁻ is an anion such as a halide, and
A is chosen from radicals of dihalides and —CH₂—CH₂—O—CH₂—CH₂—.

Such compounds are described, for instance, in European Patent Application No. 0 122 324.

(12) Quaternary polymers of vinylpyrrolidone and of vinylimidazole.

Other examples of suitable cationic polymers include, but are not limited to, cationic proteins and cationic protein hydrolysates, polyalkyleneimines, such as polyethyleneimines, polymers comprising units chosen from vinylpyridine and vinylpyridinium units, condensates of polyamines and of epichlorohydrin, quaternary polyureylenes, and chitin derivatives.

According to one embodiment of the present invention, the at least one cationic polymer is chosen from cellulose ether derivatives comprising quaternary ammonium groups, such as the products sold under the name “JR 400” by the company UNION CARBIDE CORPORATION, cationic cyclopolymers, for instance, the homo-polymers and copolymers of dimethyldiallylammonium chloride sold under the names MERQUAT® 100, MERQUAT® 550, and MERQUAT® S by the company CALGON, guar gums modified with a 2,3-epoxypropyltrimethylammonium salt, and quaternary polymers of vinylpyrrolidone and of vinylimidazole.

(13) Polyamines

As the cationic polymer, it is also possible to use (co)polyamines, which may be homopolymers or copolymers, with a plurality of amino groups. The amino group may be a primary, secondary, tertiary or quaternary amino group. The amino group may be present in a polymer backbone or a pendent group, if present, of the (co)polyamines.

As an example of the (co)polyamines, mention may be made of chitosan, (co)polyallylamines, (co)polyvinylamines, (co)polyanilines, (co)polyvinylimidazoles, (co)polydimethylaminoethylenemethacrylates, (co)polyvinylpyridines such as (co)poly-1-methyl-2-vinylpyridines, (co)polyimines such as (co) polyethyleneimines, (co)polypyridines such as (co)poly(quaternary pyridines), (co)polybiguanides such as (co)polyaminopropyl biguanides, (co)polylysines, (co)polyornithines, (co)polyarginines, (co)polyhistidines, aminodextrans, aminocelluloses, amino(co)polyvinylacetals, and salts thereof.

As the (co)polyamines, it is preferable to use (co)polylysines. Polylysine is well known. Polylysine can be a natural homopolymer of L-lysine that can be produced by bacterial fermentation. Polylysine may be α-polylysine or ε-polylysine. For example, polylysine can be ε-polylysine such as ε-Poly-L-lysine, typically used as a natural preservative in food products. Polylysine is a polyelectrolyte which is soluble in polar solvents such as water, propylene glycol and glycerol. Polylysine is commercially available in various forms, such as poly D-lysine and poly L-lysine. Polylysine can be in salt and/or solution form.

(14) Cationic Polyaminoacids

As the cationic polymer, it may be possible to use cationic polyaminoacids, which may be cationic homopolymers or copolymers, with a plurality of amino groups and carboxyl groups. The amino group may be a primary, secondary, tertiary or quaternary amino group. The amino group may be present in a polymer backbone or a pendent group, if present, of the cationic polyaminoacids. The carboxyl group may be present in a pendent group, if present, of the cationic polyaminoacids.

As examples of the cationic polyaminoacids, mention may be made of cationized collagen, cationized gelatin, stearimonium hydroxyprolyl hydrolyzed wheat protein, cocodimonium hydroxypropyl hydrolyzed wheat protein, hydroxypropyltrimonium hydrolyzed conchiolin protein, stearimonium hydroxypropyl hydrolyzed soy protein, hydroxypropyltrimonium hydrolyzed soy protein, cocodimonium hydroxypropyl hydrolyzed soy protein, and the like.

The following descriptions relate to preferable embodiments of the cationic polymer.

It may be preferable that the cationic polymer be selected from cationic starches.

As examples of the cationic starches, mention may be made of starches modified with a 2,3-epoxypropyltrimethylammonium salt (e.g. chloride), such as the product known as starch hydroxypropyltrimonium chloride according to the INC nomenclature and sold under the name SENSOMER Cl-50 from Ondeo or Pencare™ DP 1015 from Ingredion.

It may also be preferable that the cationic polymer be selected from cationic gums.

The gums may be, for example, selected from the group consisting of cassia gum, karaya gum, konjac gum, gum tragacanth, tara gum, acacia gum and gum arabic.

Examples of cationic gum include cationic polygalactomannan derivatives such as guar gum derivatives and cassia gum derivatives, e.g., CTFA: Guar Hydroxypropyltrimonium Chloride, Hydroxypropyl Guar Hydroxypropyltrimonium Chloride, and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc or the ClearHance™ from Ashland Inc.

It may also be preferable that the cationic polymer be selected from chitosans.

It may be more preferable that the cationic polymer be selected from the group consisting of 50 cyclopolymers of alkyldiallylamine and cyclopolymers of dialkyldiallylammonium such as (co)polydiallyldialkyl ammonium chloride, (co)polyamines such as (co)polylysines, cationic (co)polyaminoacids such as cationized collagen, cationic cellulose polymers, and salts thereof.

It may be even more preferable that the cationic polymer be selected from the group consisting of polylysine, polyquaternium-4, polyquaternium-10, polyquaternium-24, polyquaternium-67, starch hydroxypropyl trimonium chloride, cassia hydroxypropyltrimonium chloride, chitosan, and a mixture thereof.

The amount of the cationic polymer(s) in the composition according to the present invention may be 0.01% by weight or more, preferably 0.03% by weight or more, and more preferably 0.05% by weight or more, relative to the total weight of the composition.

The amount of the cationic polymer(s) in the composition according to the present invention may be 15% by weight or less, preferably 10% by weight or less, and more preferably 5% by weight or less, relative to the total weight of the composition.

The amount of the cationic polymer(s) in the composition according to the present invention may be from 0.01% to 15% by weight, preferably from 0.03% to 10% by weight, and more preferably from 0.05% to 5% by weight, relative to the total weight of the composition.

### (Anionic Polymer)

An anionic polymer has a positive charge density. The charge density of the anionic polymer may be from 0.1 meq/g to 20 meq/g, preferably from 1 to 15 meq/g, and more preferably from 4 to 10 meq/g if the anionic polymer is a synthetic anionic polymer, and the average substitution degree of the anionic polymer may be from 0.1 to 3.0, preferably from 0.2 to 2.7, and more preferably from 0.3 to 2.5 if the anionic polymer is a natural anionic polymer.

It may be preferable that the molecular weight of the anionic polymer be 300 or more, preferably 1,000 or more, even more preferably 5,000 or more, even more preferably 10,000 or more, even more preferably 50,000 or more, even more preferably 100,000 or more, and even more preferably 1,000,000 or more.

Unless otherwise defined in the descriptions, “molecular weight” may mean a number average molecular weight.

According to the present invention, the anionic polymer is selected from hyaluronic acid, salts thereof, and derivatives thereof.

Hyaluronic acid can be represented by the following chemical formula.

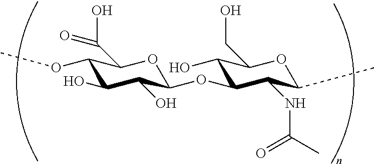

In the context of the present invention, the term “hyaluronic acid” covers in particular the basic unit of hyaluronic acid of formula:

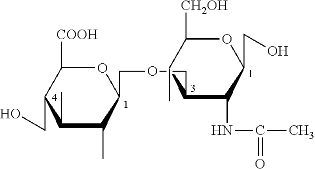

This is the smallest fraction of hyaluronic acid comprising a disaccharide dimer, namely D-glucuronic acid and N-acetylglucosamine.

The term “derivatives” of hyaluronic acid comprises, in the context of the present invention, the linear polymer comprising the polymeric unit described above, linked together in the chain via alternating P(1,4) and P(1,3) glycosidic linkages, having a molecular weight (MW) that can range between 380 and 13 000 000 daltons. This molecular weight depends in large part on the source from which the hyaluronic acid is obtained and/or on the preparation methods.

The term “salts” of hyaluronic acid comprises, in the context of the present invention, any salts of the hyaluronic acid. As the salts, mention may be made of alkaline metal salts such as sodium salts and potassium salts, alkaline earth metal salts such as magnesium salts, ammonium salts, and mixtures thereof.

In the natural state, hyaluronic acid is present in pericellular gels, in the base substance of the connective tissues of vertebrate organs such as the dermis and epithelial tissues, and in particular in the epidermis, in the synovial fluid of the joints, in the vitreous humor, in the human umbilical cord and in the crista galli apophysis.

Thus, the term “hyaluronic acid” comprises all the fractions or subunits of hyaluronic acid having a molecular weight in particular within the molecular weight range recalled above.

In the context of the present invention, hyaluronic acid fractions which do not have an inflammatory activity are preferably used.

By way of illustration of the various hyaluronic acid fractions, reference may be made to the document “Hyaluronan fragments: an information-rich system”, R. Stern et al., European Journal of Cell Biology 58 (2006) 699-715, which reviews the listed biological activities of hyaluronic acid according to its molecular weight.

According to a preferred embodiment of the present invention, the hyaluronic acid fractions suitable for the use covered by the present invention have a molecular weight of between 50 kDa and 5,000 kDa, in particular between 100 kDa and 5,000 kDa, especially between 400 kDa and 5,000 kDa. In this case, the term used is high-molecular-weight hyaluronic acid.

Alternatively, the hyaluronic acid fractions that may also be suitable for the use covered by the present invention have a molecular weight of between 50 kDa and 400 kDa. In this case, the term used is intermediate-molecular-weight hyaluronic acid.

Alternatively again, the hyaluronic acid fractions that may be suitable for the use covered by the present invention have a molecular weight of less than 50 kDa. In this case, the term used is low-molecular-weight hyaluronic acid.

It may be preferable to use a combination of two or more hyaluronic acids or salts thereof, such as a combination of a high-molecular weight hyaluronic acid or salt thereof, and an intermediate-molecular-weight hyaluronic acid or salt thereof, a combination of a high-molecular weight hyaluronic acid or salt thereof, and a low-molecular-weight hyaluronic acid or salt thereof; and a combination of an intermediate-molecular weight hyaluronic acid or salt thereof, and a low-molecular-weight hyaluronic acid or salt thereof.

Finally, the term “derivatives” of hyaluronic acid also comprises hyaluronic acid esters, in particular those in which all or some of the carboxylic groups of the acid functions are esterified with oxyethylenated alkyls or alcohols, containing from 1 to 20 carbon atoms, in particular with a degree of substitution at the level of the D-glucuronic acid of the hyaluronic acid ranging from 0.5 to 50%.

Mention may in particular be made of methyl, ethyl, n-propyl, n-pentyl, benzyl and dodecyl esters of hyaluronic acid. Such esters have in particular been described in D. Campoccia et al. “Semisynthetic resorbable materials from hyaluronan esterification”, Biomaterials 19 (1998) 2101-2127.

The hyaluronic acid derivative may be, for example, acetylated hyaluronic acid or a salt thereof.

The molecular weights indicated above are also valid for the hyaluronic acid esters.

Hyaluronic acid may in particular be hyaluronic acid supplied by the company Hyactive under the trade name CPN (MW: 10 to 150 kDa), by the company Soliance under the trade name Cristalhyal (MW: 1.1·times·10⁶), by the company Bioland under the name Nutra HA (MW: 820 000 Da), by the company Bioland under the name Nutra AF (MW: 69 000 Da), by the company Bioland under the name Oligo HA (MW: 6100 Da) or else by the company Vam Farmacos Metica under the name D Factor (MW: 380 Da).

The amount of the anionic polymer(s) in the composition according to the present invention may be 0.01% by weight or more, preferably 0.03% by weight or more, and more preferably 0.05% by weight or more, relative to the total weight of the composition.

The amount of the anionic polymer(s) in the composition according to the present invention may be 15% by weight or less, preferably 10% by weight or less, and more preferably 5% by weight or less, relative to the total weight of the composition.

The amount of the anionic polymer(s) in the composition according to the present invention may be from 0.01% to 15% by weight, preferably from 0.03% to 10% by weight, and more preferably from 0.05% to 5% by weight, relative to the total weight of the composition.

### (Amphoteric Polymer)

An amphoteric polymer has both a positive charge density and a negative charge density.

The positive charge density of the amphoteric polymer may be from 0.01 meq/g to 20 meq/g, preferably from 0.05 to 15 meq/g, and more preferably from 0.1 to 10 meq/g.

The negative charge density of the amphoteric polymer may be from 0.01 meq/g to 20 meq/g, preferably from 0.05 to 15 meq/g, and more preferably from 0.1 to 10 meq/g.

It may be preferable that the molecular weight of the amphoteric polymer be 500 or more, preferably 1,000 or more, more preferably 10,000 or more, and even more preferably 100,000 or more.

It may be preferable that the molecular weight of the amphoteric polymer be 1,000,000 or less, preferably 900,000 or less, and more preferably 800,000 or less.

Unless otherwise defined in the descriptions, “molecular weight” may mean a number average molecular weight.

According to the present invention, the amphoteric polymer is selected from cationized hyaluronic acid and salts thereof.

The cationized hyaluronic acid comprises at least one cationic group, such as an ammonium group, in the molecule thereof. The cationic group does not indicate a counter cation of the salt, because the counter cation is not in the molecule of hyaluronic acid.

As the salts, mention may be made of alkaline metal salts such as sodium salt, alkaline earth metal salts such as magnesium salts, ammonium salts, and mixtures thereof.

The cationized hyaluronic acid may have at least one quaternary ammonium group-containing group.

The cationized hyaluronic acid and/or a salt thereof may have a structure shown by the following general formula (1):

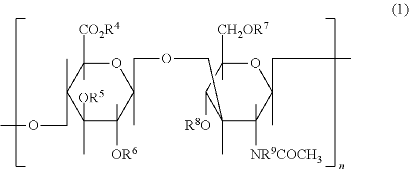

wherein
R⁴ to R⁹ individually represent a hydrogen atom or a quaternary ammonium group-containing group (excluding a case where all of R⁴ to R⁹ represent hydrogen atoms), and n represents an integer from 2 to 5,000.

Examples of the quaternary ammonium group-containing group represented by R⁴ to R⁹ in the above general formula (1) include groups shown by the following general formula (2):

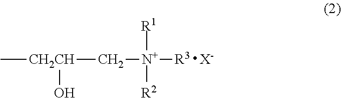

wherein
R¹ to R³ individually represent hydrocarbon groups, and X represents a monovalent anion.

Examples of the hydrocarbon groups represented by R¹ to R³ in the above general formula (2) include a linear or branched alkyl group, an unsaturated hydrocarbon group, and an aromatic hydrocarbon group. Among these, the alkyl group is preferable. Examples of the alkyl group include alkyl groups having 1 to 30 (preferably 1 to 6) carbon atoms. It is more preferable that the hydrocarbon groups represented by R¹ to R³ be alkyl groups having 1 to 3 carbon atoms.

Examples of the monovalent anion represented by X⁻ in the above general formula (2) include a halogen ion such as a fluorine ion, a bromine ion, a chlorine ion, and an iodine ion.

The quaternary ammonium group-containing group may be introduced by replacing the hydrogen atom of the carboxyl group included in hyaluronic acid and/or a salt thereof used as a raw material (hereinafter may be referred to as “raw material hyaluronic acid and/or a salt thereof”) with the quaternary ammonium group-containing group. In this case, the quaternary ammonium group-containing group is bonded to the oxygen atom of the group (—C(—O)O—) included in the cationized hyaluronic acid and/or a salt thereof according to this embodiment. The fact that the quaternary ammonium group-containing group is bonded to the oxygen atom of the group (—C(—O)O—) included in the cationized hyaluronic acid and/or a salt thereof according to this embodiment may be confirmed by the presence of a peak attributed to the carbon atom of the —C(—O)O— group to which the quaternary ammonium group-containing group is bonded via the oxygen atom, determined by analyzing the chemical shift of the nuclear magnetic resonance (¹³C NMR) spectrum.

Specifically, the quaternary ammonium group-containing group may be obtained by reacting the carboxyl group (and/or hydroxyl group) of the raw material hyaluronic acid and/or a salt thereof with a cationizing agent that contains a quaternary ammonium group. It is preferable that the cationizing agent is at least one of a 2,3-epoxypropyltrialkylammonium halide shown by the following general formula (3) and a 3-halogeno-2-hydroxypropyltrialkylammonium halide shown by the following general formula (4). The reaction of the raw material hyaluronic acid and/or a salt thereof with the cationizing agent is described in the production method hereinafter.

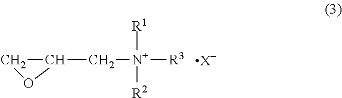

wherein
R¹ to R³ are the same as defined for the general formula (2), and X represents a halogen atom.

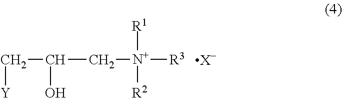

wherein
R¹ to R³ are the same as defined for the general formula (2), and X and Y individually represent halogen atoms.

Examples of the halogen atoms represented by X and Y in the above general formulas (3) and (4) include a fluorine atom, a bromine atom, a chlorine atom, and an iodine atom.

The cationized hyaluronic acid may have at least one quaternary ammonium group-containing group and has a degree of cationization of from 0.05 to 0.6, preferably from 0.1 to 0.5, and more preferably from 0.15 to 0.4.

The degree of cationization (i.e., the degree of substitution with the quaternary ammonium group-containing group) of the cationized hyaluronic acid and/or a salt thereof according to this embodiment may be determined by calculating the nitrogen content of raw material sodium hyaluronate and the nitrogen content of the cationized hyaluronic acid by the semi-micro Kjeldahl method, and calculating the degree of cationization by the following expression based on the increase in the nitrogen content.

When the nitrogen content of the raw material sodium hyaluronate is referred to as N_{N} (%) and the nitrogen content of the cationized hyaluronic acid having a degree of cationization of (x) is referred to as N_{S} (%), the relationship between the increase in the nitrogen content (N_{S}-N_{N}) and the degree of cationization (x) is shown by the following expression.

$${N}_{S} - {N}_{N} ( \% ) = [ 14 x / ( molecular weight of disaccharide unit of cationized hyaluronic acid ) ] \times 100 = [ 14 x / ( molecular weight of disaccharide unit of raw material sodium hyaluronate ) + 129.5 x ] \times 100 = [ 14 x / ( 401.3 + 129.5 x ) ] \times 100$$

Therefore, the degree of cationization (i.e., the degree of substitution with the quaternary ammonium group-containing group) can be calculated by the following expression.

$$Degree of cationization ( x ) = [ ( {N}_{S} - {N}_{N} ) \times 401.3 ] / [ 1400 - 129.5 * ( {N}_{S} - {N}_{N} ) ]$$

The degree of cationization of a cationized hyaluronic acid when a raw material hyaluronic acid is unknown may be calculated by the above expression on the assumption that the raw material sodium hyaluronate is sodium hyaluronate having a purity of 99% or more.

It is possible that 1% or more, preferably 5% or more, and more preferably 10% or more and/or 50% or less, preferably 40% or less and more preferably 30% or less of the anionic groups in hyaluronic acid be replaced with a cationic group, preferably a quaternary ammonium group-containing group, and more preferably the quaternary ammonium group-containing group represented by the above general formula (2).

As the cationized hyaluronic acid, mention may be made of hydroxypropyltrimonium hyaluronate marketed as Hyaloveil and Hyaloveul-MPF by Kewpie in Japan.

The amount of the amphoteric polymer(s) in the composition according to the present invention may be 0.01% by weight or more, preferably 0.03% by weight or more, and more preferably 0.05% by weight or more, relative to the total weight of the composition.

The amount of the amphoteric polymer(s) in the composition according to the present invention may be 15% by weight or less, preferably 10% by weight or less, and more preferably 5% by weight or less, relative to the total weight of the composition.

The amount of the amphoteric polymer(s) in the composition according to the present invention may be from 0.01% to 15% by weight, preferably from 0.03% to 10% by weight, and more preferably from 0.05% to 5% by weight, relative to the total weight of the composition.

### (Non-Polymeric Acid Having Two or More Acid Dissociation Constants)

The composition according to the present invention may include at least one non-polymeric acid having two or more pKa values or salt(s) thereof, i.e., at least one non-polymeric acid having two or more acid dissociation constants or salt(s) thereof. The pKa value (acid dissociation constant) is well known to those skilled in the art, and should be determined at a constant temperature such as 25° C.

The non-polymeric acid having two or more pKa values or salt(s) thereof can be included in the (a) polyion complex. The non-polymeric acid having two or more pKa values can function as a crosslinker, in particular an anionic crosslinker, for the cationic polymer and/or the amphoteric polymer.

It is preferable that the non-polymeric acid having two or more pKa values or salt(s) thereof be used with a cationic polymer and an anionic polymer.

The term “non-polymeric” here means that the acid is not obtained by polymerizing two or more monomers. Therefore, the non-polymeric acid does not correspond to an acid obtained by polymerizing two or more monomers such as polycarboxylic acid.

It is preferable that the molecular weight of the non-polymeric acid having two or more pKa values or salt(s) thereof is 1000 or less, preferably 800 or less, and more preferably 700 or less.

There is no limit to the type of the non-polymeric acid having two or more pKa values or salt(s) thereof. Two or more different types of non-polymeric acids having two or more pKa values or salts thereof may be used in combination. Thus, a single type of a non-polymeric acid having two or more pKa values or a salt thereof or a combination of different types of non-polymeric acids having two or more pKa values or salts thereof may be used.

The term “salt” here means a salt formed by addition of suitable base(s) to the non-polymeric acid having two or more pKa values, which may be obtained from a reaction with the non-polymeric acid having two or more pKa values with the base(s) according to methods known to those skilled in the art. As the salt, mention may be made of metal salts, for example salts with alkaline metals such as Na and K, and salts with alkaline earth metals such as Mg and Ca, and ammonium salts.

The non-polymeric acid having two or more pKa values or salt(s) thereof may be an organic acid or salt(s) thereof, and preferably a hydrophilic or water-soluble organic acid or salt(s) thereof.

The non-polymeric acid having two or more pKa values may have at least two acid groups selected from the group consisting of a carboxylic group, a sulfuric group, a sulfonic group, a phosphoric group, a phosphonic group, a phenolic hydroxyl group, and a mixture thereof.

The non-polymeric acid having two or more pKa values may be a non-polymeric polyvalent acid.

The non-polymeric acid having two or more pKa values may be selected from the group consisting of dicarboxylic acids, disulfonic acids, and diphosphoric acids, and a mixture thereof.

The non-polymeric acid having two or more pKa values or salt(s) thereof may be selected from the group consisting of oxalic acid, malonic acid, succinic acid, glutaric acid, adipic acid, pimelic acid, suberic acid, azelaic acid, sebacic acid, fumaric acid, maleic acid, malic acid, citric acid, aconitic acid, oxaloacetic acid, tartaric acid, and salts thereof; aspartic acid, glutamic acid, and salts thereof; terephthalylidene dicamphor sulfonic acid or salts thereof (Mexoryl SX), Benzophenone-9; phytic acid, and salts thereof; Red 2 (Amaranth), Red 102 (New Coccine), Yellow 5 (Tartrazine), Yellow 6 (Sunset Yellow FCF), Green 3 (Fast Green FCF), Blue 1 (Brilliant Blue FCF), Blue 2 (Indigo Carmine), Red 201 (Lithol Rubine B), Red 202 (Lithol Rubine BCA), Red 204 (Lake Red CBA), Red 206 (Lithol Red CA), Red 207 (Lithol Red BA), Red 208 (Lithol Red SR), Red 219 (Brilliant Lake Red R), Red 220 (Deep Maroon), Red 227 (Fast Acid Magenta), Yellow 203 (Quinoline Yellow WS), Green 201 (Alizanine Cyanine Green F), Green 204 (Pyranine Conc), Green 205 (Light Green SF Yellowish), Blue 203 (Patent Blue CA), Blue 205 (Alfazurine FG), Red 401 (Violamine R), Red 405 (Permanent Re F5R), Red 502 (Ponceau 3R), Red 503 (Ponceau R), Red 504 (Ponceau SX), Green 401 (Naphtol Green B), Green 402 (Guinea Green B), and Black 401 (Naphtol Blue Black); folic acid, ascorbic acid, erythorbic acid, and salts thereof; cystine and salts thereof; EDTA and salts thereof; glycyrrhizin and salts thereof; and a mixture thereof.

It may be preferable that the non-polymeric acid having two or more pKa values or salt(s) thereof be selected from the group consisting of terephthalylidene dicamphor sulfonic acid and salts thereof (Mexoryl SX), Yellow 6 (Sunset Yellow FCF), ascorbic acid, phytic acid and salts thereof, and a mixture thereof.

The amount of the non-polymeric acid having two or more pKa values or salt(s) thereof in the composition according to the present invention may be 0.001% by weight or more, preferably 0.005% by weight or more, and more preferably 0.01% by weight or more, relative to the total weight of the composition.

The amount of the non-polymeric acid having two or more pKa values or salt(s) thereof in the composition according to the present invention may be 15% by weight or less, preferably 10% by weight or less, and more preferably 5% by weight or less, relative to the total weight of the composition.

The amount of the non-polymeric acid having two or more pKa values or salt(s) thereof in the composition according to the present invention may be from 0.001% to 15% by weight, preferably from 0.005% to 10% by weight, and more preferably from 0.01% to 5% by weight, relative to the total weight of the composition.

### (Non-Polymeric Base Having Two or More Base Dissociation Constants)

The composition according to the present invention may include at least one non-polymeric base having two or more pKb values or salt(s) thereof, i.e., at least one non-polymeric base having two or more base dissociation constants or salt(s) thereof. The pKb value (base dissociation constant) is well known to those skilled in the art, and should be determined at a constant temperature such as 25° C.

The non-polymeric base having two or more pKb values or salt(s) thereof can be included in the (a) polyion complex. The non-polymeric base having two or more pKb values can function as a crosslinker, in particular a cationic crosslinker, for the anionic polymer and/or the amphoteric polymers.

It is preferable that the non-polymeric acid having two or more pKb values or salt(s) thereof be used with a cationic polymer and an anionic polymer.

The term “non-polymeric” here means that the base is not obtained by polymerizing two or more monomers. Therefore, the non-polymeric base does not correspond to a base obtained by polymerizing two or more monomers such as polyallylamine.

It is preferable that the molecular weight of the non-polymeric base having two or more pKb values or salt(s) thereof be 1000 or less, preferably 800 or less, and more preferably 700 or less.

There is no limit to the type of the non-polymeric base having two or more pKb values or salt(s) thereof. Two or more different types of non-polymeric bases having two or more pKb values or salts thereof may be used in combination. Thus, a single type of a non-polymeric base having two or more pKb values or a salt thereof or a combination of different types of non-polymeric bases having two or more pKb values or salts thereof may be used.

The term “salt” here means a salt formed by addition of suitable acid(s) to the non-polymeric base having two or more pKb values, which may be obtained from a reaction with the non-polymeric base having two or more pKb values with the acid(s) according to methods known to those skilled in the art. As the salt, mention may be made of ammonium salts, for example salts with inorganic acids such as HCl and HNO₃, and salts with organic acids such as carboxylic acids and sulfonic acids.

The non-polymeric base having two or more pKb values or salt(s) thereof may be an organic base or salt(s) thereof, and preferably a hydrophilic or water-soluble organic base or salt(s) thereof.

The non-polymeric base having two or more pKb values may have at least two basic groups selected from the group consisting of an amino group, a guanidine group, a biguanide group, an imidazole group, an imino group, a pyridyl group and a mixture thereof.

The non-polymeric base having two or more pKb values may be selected from the group consisting of non-polymeric diamines such as ethylenediamine, propylenediamine, pentanediamine, hexanediamine, urea and derivatives thereof and guanidine and derivatives thereof, non-polymeric polyamines such as spermine and spermidine, basic amino acids, and a mixture thereof.

The non-polymeric base having two or more pKb values or salt(s) thereof may be selected from the group consisting of arginine, lysine, histidine, cysteine, cystine, tyrosine, tryptophan, ornithine, and a mixture thereof.

It may be preferable that the non-polymeric base having two or more pKb values or salt(s) thereof be selected from the group consisting of arginine, lysine, histidine, and a mixture thereof.

The amount of the non-polymeric base having two or more pKb values or salt(s) thereof in the composition according to the present invention may be 0.001% by weight or more, preferably 0.005% by weight or more, and more preferably 0.01% by weight or more, relative to the total weight of the composition.

The amount of the non-polymeric base having two or more pKb values or salt(s) thereof in the composition according to the present invention may be 15% by weight or less, preferably 10% by weight or less, and more preferably 5% by weight or less, relative to the total weight of the composition.

The amount of the non-polymeric base having two or more pKb values or salt(s) thereof in the composition according to the present invention may be from 0.001% to 15% by weight, preferably from 0.005% to 10% by weight, and more preferably from 0.01% to 5% by weight, relative to the total weight of the composition.

### [Hydrophilic Antioxidant]

The composition according to the present invention may include, as the ingredient (b), at least one hydrophilic antioxidant. Two or more hydrophilic antioxidants may be used in combination. Thus, a single type of hydrophilic antioxidant or a combination of different types of hydrophilic antioxidants may be used.

According to the present invention, antioxidant agents are compounds or substances that can scavenge the various radical forms which may be present in the skin; preferably, they simultaneously scavenge all the various radical forms present.

The (b) hydrophilic antioxidant agent means that the partition coefficient of the antioxidant agent between n-butanol and water is <1, more preferably <0.5 and even more preferably <0.1.

It is preferable that the (b) hydrophilic antioxidant be selected from ascorbic acid, derivatives thereof, salts thereof, and mixtures thereof.

As derivatives of ascorbic acid, mention may be made of, for example, ascorbyl glucoside. The term “ascorbyl glucoside” is understood to mean the condensation product of glucose, in the D form, that is to say in the α- or β-glucopyranose or α- or β-furanose form, or in the L form, with ascorbic acid, preferably in the L form. L-Ascorbic acid 2-O-α-D-glucopyranoside may be preferred for use in the present invention. It is available in particular from Hayashibara.

As examples of derivatives of ascorbic acid, mention may also be made of ethers of ascorbic acid. The ethers of ascorbic acid may be alkyl ethers of ascorbic acid, such as ethyl ascorbic acid, in particular 3-O-ethyl ascorbic acid.

As salts of ascorbic acid, mention may be made of, metal salts of ascorbic acid, for instance, sodium ascorbate, magnesium ascorbyl phosphate and sodium ascorbyl phosphate.

It may be preferable that the (b) hydrophilic antioxidant be selected from the group consisting of ascorbic acid, ascorbyl glycoside, ethyl ascorbic acid, sodium ascorbate, magnesium ascorbyl phosphate, sodium ascorbyl phosphate, and a mixture thereof.

The amount of the (b) hydrophilic antioxidant(s) in the composition according to the present invention may be 0.01% by weight or more, preferably 0.05% by weight or more, and more preferably 0.1% by weight or more, relative to the total weight of the composition.

The amount of the (b) hydrophilic antioxidant(s) in the composition according to the present invention may be 30% by weight or less, preferably 25% by weight or less, and more preferably 20% by weight or less, relative to the total weight of the composition.

The amount of the (b) hydrophilic antioxidant(s) in the composition according to the present invention may be from 0.01% to 30% by weight, preferably from 0.05% to 25% by weight, and more preferably from 0.1% to 20% by weight, relative to the total weight of the composition.

### [Lipophilic Antioxidant]

The composition according to the present invention may include, as the ingredient (b), at least one lipophilic antioxidant. Two or more lipophilic antioxidants may be used in combination.

Thus, a single type of lipophilic antioxidant or a combination of different types of lipophilic antioxidants may be used.

According to the present invention, antioxidant agents are compounds or substances that can scavenge the various radical forms which may be present in the skin; preferably, they simultaneously scavenge all the various radical forms present.

The (b) lipophilic antioxidant means that the partition coefficient of the antioxidant agent between n-butanol and water is >1, more preferably >10 and even more preferably >100.

The (b) lipophilic antioxidant is hydrophobic, and is not a hydrophilic antioxidant such as ascorbic acid or glutathione.

As the (b) lipophilic antioxidant, mention may be made of phenolic antioxidants which have a hindered phenol structure or a semi-hindered phenol structure within the molecule. As specific examples of such compounds, mention may be made of 3,5-bis(1,1-dimethylethyl)-4-hydroxybenzenepropanoic acid) which has the INCI name of pentaerythrityl tetra-di-t-butyl hydroxyhydrocinnamate, 2,6-di-tert-butyl-4-methylphenol, 2,6-di-tert-butyl-4-ethylphenol, mono- or di- or tri-(α-methylbenzyl)phenol, 2,2′-methylenebis(4-ethyl-6-tert-butylphenol), 2,2′-methylenebis(4-methyl-6-tert-butylphenol), 4,4′-butylidenebis(3-methyl-6-tert-butylphenol), 4,4′-thiobis(3-methyl-6-tert-butylphenol), 2,5-di-tert-butylhydroquinone, 2,5-di-tert-amylhydroquinone, tris[N-(3,5-di-tert-butyl-4-hydroxybenzyl)]isocyanurate, 1,1,3-tris(2-methyl-4-hydroxy-5-tert-butylphenyl)butane, butylidene-1,1bis[3-(3-tert-butyl-4-hydroxy-5-methylphenyl)propionate], octadecyl 3-(3,5-di-tert-butyl-4-hydroxyphenyl)propionate, tetrakis[methylene-3-(3,5-di-tert-butyl-4-hydroxyphenyl)propionato]methane, triethylene glycol bis[3-(3-tert-butyl-4-hydroxy-5-methylphenyl)propionate], 3,9-bis{2-[3-(3-tert-butyl-4-hydroxy-5-methylphenyl)propionyloxy]-1,1-dimethylethyl}-2,4,8,10-tetraoxaspiro [5.5]undecane, 1,3,5-trimethyl-2,4,6-tris(3,5-di-tert-butyl-4-hydroxybenzyl)benzene, 2,2-thiodiethylenebis[3-(3,5-di-tert-butyl-4-hydroxyphenyl)propionate], N,N′-hexamethylenebis(3,5-di-tert-butyl-4-hydroxyhydrocinnamide), 1,6-hexanediol bis[3-(3,5-di-tert-butyl-4-hydroxyphenyl)propionate], 1,3,5-tris[(4-tert-butyl-3-hydroxy-2,6-xylyl)methyl]-1,3,5-triazine-2,4,6-trione, 2,4-bis(n-octylthio)-6-(4-hydroxy-3,5-di-tert-butylanilino)-1,3,5-triazine, 2-tert-butyl-6-(3′-tert-butyl-5′-methyl-2′-hydroxybenzyl)-4-methylphenyl acrylate, 2-[1-(2-hydroxy-3,5-di-tert-pentylphenyl)ethyl]-4,6-di-tert-pentylphenyl acrylate, 4,6-bis[(octylthio)methyl]-o-cresol, 2,4-di-tert-butylphenyl-3,5-di-tert-butyl-4-hydroxybenzoate and 1,6-hexanediolbis[3-(3,5-di-tert-butyl-4-hydroxyphenyl) propionate].

As the (b) lipophilic antioxidant, mention may be made of BHA (butylated hydroxyl anisole) and BHT (butylated hydroxyl toluene), vitamin E (or tocopherols and tocotrienol) and derivatives thereof, such as the phosphate derivative, for instance TPNA® sold by the company Showa Denko, coenzyme Q10 (or ubiquinone), idebenone, certain carotenoids such as lutein, astaxanthin, beta-carotene, polyphenols, phenolic acids and derivatives (e. g., chlorogenic acid), and flavonoids, which represent the main subgroup of polyphenols.

Among the flavonoids, mention may be made especially of chalcones, hydroxylated chalcones and reduced derivatives thereof (as described especially in patent FR 2 608 150), for instance phloretin, neohesperidin, phloridzin, aspalathin, etc., flavanones, for instance hesperetin and naringin, flavonols, for instance quercetin, rutin, flavanols, for instance catechin, EGCG, flavones, for instance apigenidin, and finally anthocyans. Mention may also be made of tannins. Reference may also be made to the compounds described in patent applications FR 2 699 818, FR 2 706 478, FR 2 907 339, FR 2 814 943 and FR 2 873 026.

Polyphenol compounds may especially be derived from plant extracts chosen from extracts of green tea, apple, hop, guava, cocoa, or wood such as chestnut, oak, horse chestnut or hazel. It is also possible to use an extract of pinaster bark, for example, that obtained according to processes described in U.S. Pat. Nos. 4,698,360, 6,372,266 and 5,720,956. As examples of such extracts, the compound referenced by the INCI name pinus pinaster (bark extract) and by the CTFA name pine (pinus pinaster) bark extract may be cited. It may, in particular, be the extract of pinaster bark marketed under the name PYCNOGENOL® by the BIOLANDES AROMES firm and/or HORPHAG Research. The extracts of (Maritime) pine bark from LAYN Natural Ingredients, Pine Bark from Blue California, and also Oligopin® from D.R.T. (Les Derives Resiniques et Terpeniques) may also be cited.

In the context of the present invention, the term “polyphenol compound” thus also covers the plant extract itself, rich in these polyphenol compounds.

The (b) lipophilic antioxidant that may also be mentioned includes dithiolanes, for instance asparagusic acid, or derivatives thereof, for instance siliceous dithiolane derivatives, especially such as those described in patent application FR 2 908 769.

The (b) lipophilic antioxidant that may also be mentioned includes:
glutathione and derivatives thereof (GSH and/or GSHOEt), such as glutathione alkyl esters (such as those described in patent applications FR 2 704 754 and FR 2 908 769); and cysteine and derivatives thereof, such as N-acetylcysteine or L-2-oxothiazolidine-4-carboxylic acid. Reference may also be made to the cysteine derivatives described in patent applications FR 2 877 004 and FR 2 854 160; and
ferulic acid and derivatives thereof (esters, salts, etc.). Mention may particularly be made of esters of ferulic acid and of C₁-C₃₀ alcohols, in particular methyl ferulate, ethyl ferulate, isopropyl ferulate, octyl ferulate and oryzanyl ferulate;
certain enzymes for defending against oxidative stress, such as catalase, superoxide dismutase (SOD), lactoperoxidase, glutathione peroxidase and quinone reductases;
benzylcyclanones; substituted naphthalenones; pidolates (as described especially in patent application EP 0 511 118); caffeic acid and derivatives thereof, gamma-oryzanol; melatonin, sulforaphane and extracts containing it (excluding cress);
the diisopropyl ester of N,N′-bis(benzyl)ethylenediamine-N,N′-diacetic acid, as described especially in patent applications WO 94/11338, FR 2 698 095, FR 2 737 205 or EP 0 755 925; and deferoxamine (or desferal) as described in patent application FR 2 825 920.

The (b) lipophilic antioxidants that are preferably used are the chalcones, more particularly phloretin or neohesperidin, the diisopropyl ester of N,N′-bis(benzyl)ethylenediamine-N,N′-diacetic acid, or an extract of pinaster bark such as PYCNOGENOL®.

As examples of the (b) lipophilic antioxidant, mention may be made of pentaerythrityl tetra-di-t-butyl hydroxyhydrocinnamate, nordihydroguaiaretic acid, propyl gallate, butylated hydroxyl toluene, butylated hydroxyl anisole, ascorbyl palmitate, tocopherol, and mixtures thereof.

The amount of the (b) lipophilic antioxidant(s) in the composition according to the present invention may be 0.001% by weight or more, preferably 0.005% by weight or more, and more preferably 0.01% by weight or more, relative to the total weight of the composition.

The amount of the (b) lipophilic antioxidant(s) in the composition according to the present invention may be 15% by weight or less, preferably 10% by weight or less, and more preferably 5% by weight or less, relative to the total weight of the composition.

The amount of the (b) lipophilic antioxidant(s) in the composition according to the present invention may be from 0.001% to 15% by weight, preferably from 0.005% to 10% by weight, and more preferably from 0.01% to 5% by weight, relative to the total weight of the composition.

### [Water]

The composition according to the present invention comprises (c) water.

The amount of the (c) water may be 10% by weight or more, preferably 30% by weight or more, and more preferably 50% by weight or more, relative to the total weight of the composition.

The amount of the (c) water may be 99% by weight or less, preferably 97% by weight or less, and more preferably 95% by weight or less, relative to the total weight of the composition.

The amount of the (c) water may be from 10% to 99% by weight, preferably from 30% to 97% by weight, and more preferably from 50% to 95% by weight, relative to the total weight of the composition.

### [Surfactant]

The composition according to the present invention may further include (d) at least one surfactant. Two or more surfactants may be used in combination. Thus, a single type of surfactant or a combination of different types of surfactants may be used.

The surfactant used in the present invention may be selected from the group consisting of anionic surfactants, amphoteric surfactants, cationic surfactants and nonionic surfactants.

### (Anionic Surfactants)

The composition according to the present invention may comprise at least one anionic surfactant. Two or more anionic surfactants may be used in combination.

It is preferable that the anionic surfactant be selected from the group consisting of (C₆-C₃₀)alkyl sulfates, (C₆-C₃₀)alkyl ether sulfates, (C₆-C₃₀)alkylamido ether sulfates, alkylaryl polyether sulfates, monoglyceride sulfates; (C₆-C₃₀)alkylsulfonates, (C₆-C₃₀)alkylamide sulfonates, (C₆-C₃₀)alkylaryl sulfonates, α-olefin sulfonates, paraffin sulfonates; (C₆-C₃₀)alkyl phosphates; (C₆-C₃₀)alkyl sulfosuccinates, (C₆-C₃₀)alkyl ether sulfosuccinates, (C₆-C₃₀)alkylamide sulfosuccinates; (C₆-C₃₀)alkyl sulfoacetates; (C₆-C₂₄)acyl sarcosinates; (C₆-C₂₄)acyl glutamates; (C₆-C₃₀)alkylpolyglycoside carboxylic ethers; (C₆-C₃₀)alkylpolyglycoside sulfosuccinates; (C₆-C₃₀)alkyl sulfosuccinamates; (C₆-C₂₄)acyl isethionates; N—(C₆-C₂₄)acyl taurates; C₆-C₃₀ fatty acid salts; coconut oil acid salts or hydrogenated coconut oil acid salts; (C₈-C₂₀)acyl lactylates; (C₆-C₃₀)alkyl-D-galactoside uronic acid salts; polyoxyalkylenated (C₆-C₃₀)alkyl ether carboxylic acid salts; polyoxyalkylenated (C₆-C₃₀)alkylaryl ether carboxylic acid salts; and polyoxyalkylenated (C₆-C₃₀)alkylamido ether carboxylic acid salts; and corresponding acid forms.

In at least one embodiment, the anionic surfactants are in the form of salts such as salts of alkali metals, for instance sodium; salts of alkaline-earth metals, for instance magnesium; ammonium salts; amine salts; and amino alcohol salts. Depending on the conditions, they may also be in acid form.

It is more preferable that the anionic surfactant be selected from salts of (C₆-C₃₀)alkyl sulfate, (C₆-C₃₀)alkyl ether sulfates or polyoxyalkylenated (C₆-C₃₀)alkyl ether carboxylic acid salified 50 or not.

### (Amphoteric Surfactants)

The composition according to the present invention may comprise at least one amphoteric surfactant. Two or more amphoteric surfactants may be used in combination.

The amphoteric or zwitterionic surfactants can be, for example (non-limiting list), amine derivatives such as aliphatic secondary or tertiary amines, and optionally quaternized amine derivatives, in which the aliphatic radical is a linear or branched chain including 8 to 22 carbon atoms and containing at least one water-solubilizing anionic group (for example, carboxylate, sulphonate, sulphate, phosphate or phosphonate).

The amphoteric surfactant may preferably be selected from the group consisting of betaines and amidoaminecarboxylated derivatives.

It is preferable that the amphoteric surfactant be selected from betaine-type surfactants.

The betaine-type amphoteric surfactant is preferably selected from the group consisting of alkylbetaines, alkylamidoalkylbetaines, sulfobetaines, phosphobetaines, and alkylamidoalkylsulfobetaines, in particular, (C₈-C₂₄)alkylbetaines, (C₈-C₂₄)alkylamido(C₁-C₈)alkylbetaines, sulphobetaines, and (C₈-C₂₄)alkylamido(C₁-C₅)alkylsulphobetaines. In one embodiment, the amphoteric surfactants of betaine type are chosen from (C₈-C₂₄)alkylbetaines, (C₈-C₂₄)alkylamido(C₁-C₈)alkylsulphobetaines, sulphobetaines, and phosphobetaines.

Non-limiting examples that may be mentioned include the compounds classified in the CTFA International Cosmetic Ingredient Dictionary & Handbook, 15th Edition, 2014, under the names cocobetaine, laurylbetaine, cetylbetaine, coco/oleamidopropylbetaine, cocamidopropylbetaine, palmitamidopropylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular cocobetaine and cocamidopropylbetaine.

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

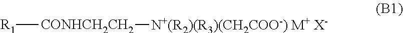

in which:
R₁ denotes an alkyl radical of an acid R₁—COOH present in hydrolysed coconut oil, a heptyl, nonyl or undecyl radical,
R₂ denotes a beta-hydroxyethyl group,
R₃ denotes a carboxymethyl group,
M⁺ denotes a cationic ion derived from alkaline metals such as sodium; ammonium ion; or an ion derived from an organic amine;
X⁻ denotes an organic or inorganic anionic ion such as halides, acetates, phosphates, nitrates, alkyl(C₁-C₄)sulfates, alkyl(C₁-C₄)— or alkyl(C₁-C₄)aryl-sulfonates, particularly methylsulfate and ethylsulfate; or M⁺ and X⁻ are not present;

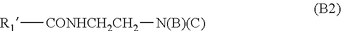

in which:
R₁′ denotes an alkyl radical of an acid R₁′—COOH present in coconut oil or in hydrolysed linseed oil, an alkyl radical, such as a C₇, C₉, C₁₁ or C₁₃ alkyl radical, a C₁₇ alkyl radical and its iso-form, or an unsaturated C₁₇ radical,
B represents —CH₂CH₂OX′,
C represents —(CH₂)_{z}Y′, with z=1 or 2,
X′ denotes a —CH₂—COOH group, —CH₂—COOZ′, —CH₂CH₂—COOH, —CH₂CH₂—COOZ′ or a hydrogen atom, and
Y′ denotes —COOH, —COOZ′, —CH₂—CHOH—SO₃Z′, —CH₂—CHOH—SO₃H radical or a —CH₂—CH(OH)—SO₃—Z′ radical,
wherein Z′ represents an ion of an alkaline or alkaline earth metal such as sodium, an ion derived from an organic amine or an ammonium ion;
and

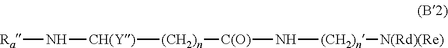

in which:
Y″ denotes —C(O)OH, —C(O)OZ″, —CH₂—CH(OH)—SO₃H or —CH₂—CH(OH)—SO₃—Z″, wherein
Z″ denotes a cationic ion derived from alkaline metal or alkaline-earth metals such as sodium, an ion derived from an organic amine or an ammonium ion;
Rd and Re denote a C₁-C₄ alkyl or C₁-C₄ hydroxyalkyl radical;
Rₐ″ denotes a C₁₀-C₃₀ group alkyl or alkenyl group from an acid, and
n and n′ independently denote an integer from 1 to 3.

It is preferable that the amphoteric surfactant with formula B1 and B2 be selected from (C₈-C₂₄)-alkyl amphomonoacetates, (C₈-C₂₄)alkyl amphodiacetates, (C₈-C₂₄)alkyl amphomonopropionates, and (C₈-C₂₄)alkyl amphodipropionates.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C₂M concentrate by the company Rhodia Chimie.

Among the compounds of formula (B′2) mention may be made of sodium diethylaminopropyl cocoaspartamide (CTFA) marketed by CHIMEX under the denomination CHIMEXANE HB.

### (Cationic Surfactants)

The composition according to the present invention may comprise at least one cationic surfactant. Two or more cationic surfactants may be used in combination.

The cationic surfactant may be selected from the group consisting of optionally polyoxyalkylenated, primary, secondary or tertiary fatty amine salts, quaternary ammonium salts, and mixtures thereof.

Examples of quaternary ammonium salts that may be mentioned include, but are not limited to:
those of general formula (B3) below:

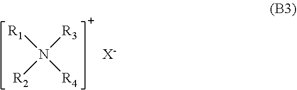

wherein
R₁, R₂, R₃, and R₄, which may be identical or different, are chosen from linear and branched aliphatic radicals including from 1 to 30 carbon atoms and optionally including heteroatoms such as oxygen, nitrogen, sulfur and halogens. The aliphatic radicals may be chosen, for example, from alkyl, alkoxy, C₂-C₆ polyoxyalkylene, alkylamide, (C₁₂-C₂₂)alkylamido(C₂-C₆)alkyl, (C₁₂-C₂₂)alkylacetate and hydroxyalkyl radicals; and aromatic radicals such as aryl and alkylaryl; and X⁻ is chosen from halides, phosphates, acetates, lactates, (C₂-C₆) alkyl sulfates and alkyl- or alkylaryl-sulfonates;
quaternary ammonium salts of imidazoline, for instance those of formula (B4) below:

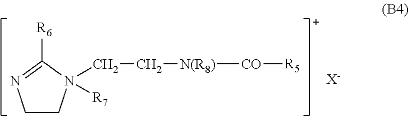

wherein:
R₅ is chosen from alkenyl and alkyl radicals including from 8 to 30 carbon atoms, for example fatty acid derivatives of tallow or of coconut;
R₆ is chosen from hydrogen, C₁-C₄ alkyl radicals, and alkenyl and alkyl radicals including from 8 to 30 carbon atoms;
R₇ is chosen from C₁-C₄ alkyl radicals;
R₈ is chosen from hydrogen and C₁-C₄ alkyl radicals; and
X⁻ is chosen from halides, phosphates, acetates, lactates, alkyl sulfates, alkyl sulfonates, and alkylaryl sulfonates. In one embodiment, R₅ and R₆ are, for example, a mixture of radicals chosen from alkenyl and alkyl radicals including from 12 to 21 carbon atoms, such as fatty acid derivatives of tallow, R₇ is methyl and R₅ is hydrogen. Examples of such products include, but are not limited to, Quaternium-27 (CTFA 1997) and Quaternium-83 (CTFA 1997), which are sold under the names “Rewoquat®” W75, W90, W75PG and W75HPG by the company Witco;
Di or tri quaternary ammonium salts of formula (B5):

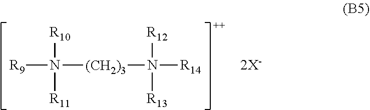

wherein:
R₉ is chosen from aliphatic radicals including from 16 to 30 carbon atoms;
R₁₀ is chosen from hydrogen or alkyl radicals including from 1 to 4 carbon atoms or a group —(CH₂)₃ (R₁₆ₐ)(R₁₇ₐ)(R₁₈ₐ)N⁺X—;
R₁₁, R₁₂, R₁₃, R₁₄, R₁₆ₐ, R₁₇ₐ, and R₁₈ₐ, which may be identical or different, are chosen from hydrogen and alkyl radicals including from 1 to 4 carbon atoms; and
X⁻ is chosen from halides, acetates, phosphates, nitrates, ethyl sulfates, and methyl sulfates.

An example of one such diquaternary ammonium salt is FINQUAT CT-P of FINETEX (Quaternium-89) or FINQUAT CT (Quaternium-75);
and
quaternary ammonium salts including at least one ester function, such as those of formula (B6) below:

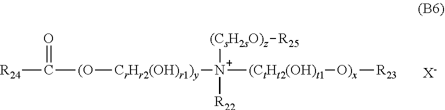

wherein:
R₂₂ is chosen from C₁-C₆ alkyl radicals and C₁-C₆ hydroxyalkyl and dihydroxyalkyl radicals;
R₂₃ is chosen from:
the radical below:

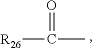

linear and branched, saturated and unsaturated C₁-C₂₂ hydrocarbon-based radicals R₂₇, and hydrogen,
R₂₅ is chosen from:
the radical below:

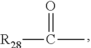

linear and branched, saturated and unsaturated C₁-C₆ hydrocarbon-based radicals R₂₉, and hydrogen,
R₂₄, R₂₆, and R₂₈, which may be identical or different, are chosen from linear and branched, saturated and unsaturated, C₇-C₂₁, hydrocarbon-based radicals;
r, s, and t, which may be identical or different, are chosen from integers ranging from 2 to 6; each of r1 and t1, which may be identical or different, is 0 or 1, and r2+r1=2r and t1+2t=2t;
y is chosen from integers ranging from 1 to 10;
x and z, which may be identical or different, are chosen from integers ranging from 0 to 10;
X⁻ is chosen from simple and complex, organic and inorganic anions; with the proviso that the sum x+y+z ranges from 1 to 15, that when x is 0, R₂₃ denotes R₂₇, and that when z is 0, R₂₅ denotes R₂₉. R₂₂ may be chosen from linear and branched alkyl radicals. In one embodiment, R₂₂ is chosen from linear alkyl radicals. In another embodiment, R₂₂ is chosen from methyl, ethyl, hydroxyethyl, and dihydroxypropyl radicals, for example methyl and ethyl radicals. In one embodiment, the sum x+y+z ranges from 1 to 10. When R₂₃ is a hydrocarbon-based radical R₂₇, it may be long and include from 12 to 22 carbon atoms, or short and include from 1 to 3 carbon atoms. When R₂₅ is a hydrocarbon-based radical R₂₉, it may include, for example, from 1 to 3 carbon atoms. By way of a non-limiting example, in one embodiment, R₂₄, R₂₆, and R₂₈, which may be identical or different, are chosen from linear and branched, saturated and unsaturated, C₁₁-C₂₁ hydrocarbon-based radicals, for example from linear and branched, saturated and unsaturated C₁₁-C₂₁ alkyl and alkenyl radicals. In another embodiment, x and z, which may be identical or different, are 0 or 1. In one embodiment, y is equal to 1. In another embodiment, r, s and t, which may be identical or different, are equal to 2 or 3, for example equal to 2. The anion X⁻ may be chosen from, for example, halides, such as chloride, bromide, and iodide; and C₁-C₄ alkyl sulfates, such as methyl sulfate. However, methanesulfonate, phosphate, nitrate, tosylate, an anion derived from an organic acid, such as acetate and lactate, and any other anion that is compatible with the ammonium including an ester function, are other non-limiting examples of anions that may be used according to the present invention. In one embodiment, the anion X⁻ is chosen from chloride and methyl sulfate.

In another embodiment, the ammonium salts of formula (B6) may be used, wherein:
R₂₂ is chosen from methyl and ethyl radicals,
x and y are equal to 1;
z is equal to 0 or 1;
r, s and t are equal to 2;
R₂₃ is chosen from:
the radical below:

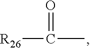

methyl, ethyl, and C₁₄-C₂₂ hydrocarbon-based radicals, and hydrogen;
R₂₅ is chosen from:
the radical below:

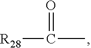

an hydrogen;
R₂₄, R₂₆, and R₂₈, which may be identical or different, are chosen from linear and branched, saturated and unsaturated, C₁₃-C₁₇ hydrocarbon-based radicals, for example from linear and branched, saturated and unsaturated, C₁₃-C₁₇ alkyl and alkenyl radicals.

In one embodiment, the hydrocarbon-based radicals are linear.

Non-limiting examples of compounds of formula (B6) that may be mentioned include salts, for example chloride and methyl sulfate, of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethyl-ammonium, and mixtures thereof. In one embodiment, the acyl radicals may include from 14 to 18 carbon atoms, and may be derived, for example, from a plant oil, for instance palm oil and sunflower oil. When the compound includes several acyl radicals, these radicals may be identical or different.

These products may be obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification may be followed by a quaternization using an alkylating agent chosen from alkyl halides, for example methyl and ethyl halides; dialkyl sulfates, for example dimethyl and diethyl sulfates; methyl methanesulfonate; methyl para-toluenesulfonate; glycol chlorohydrin; and glycerol chlorohydrin.

Such compounds are sold, for example, under the names Dehyquart® by the company Cognis, Stepanquat® by the company Stepan, Noxamium® by the company Ceca, and “Rewoquat® WE 18” by the company Rewo-Goldschmidt.

Other non-limiting examples of ammonium salts that may be used in the composition according to the present invention include the ammonium salts including at least one ester function described in U.S. Pat. Nos. 4,874,554 and 4,137,180.

The quaternary ammonium salts mentioned above that may be used in the composition according to the present invention include, but are not limited to, those corresponding to formula (I), for example tetraalkylammonium chlorides, for instance dialkyldimethylammonium and alkyltrimethylammonium chlorides in which the alkyl radical includes from about 12 to 22 carbon atoms, such as behenyltrimethylammonium, distearyldimethylammonium, cetyltrimethylammonium and benzyldimethylstearylammonium chloride; palmitylamidopropyltrimethylammonium chloride; and stearamidopropyldimethyl(myristyl acetate)ammonium chloride, sold under the name “Ceraphyl® 70” by the company Van Dyk.

According to one embodiment, the cationic surfactant that may be used in the composition according to the present invention is chosen from behenyltrimethylammonium chloride, cetyltrimethylammonium chloride, Quaternium-83, Quaternium-87, Quaternium-22, behenylamidopropyl-2,3-dihydroxypropyldimethylammonium chloride, palmitylamidopropyltrimethylammonium chloride, and stearamidopropyldimethylamine.

### (Nonionic Surfactants)

The composition according to the present invention may comprise at least one nonionic surfactant. Two or more nonionic surfactants may be used in combination.

The nonionic surfactants are compounds well known in and of themselves (see, for example, in this regard, “Handbook of Surfactants” by M. R. Porter, Blackie & Son publishers (Glasgow and London), 1991, pp. 116-178). Thus, they can, for example, be chosen from alcohols, alpha-diols, alkylphenols and esters of fatty acids, these compounds being ethoxylated, propoxylated or glycerolated and having at least one fatty chain comprising, for example, from 8 to 30 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils of plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives; amine oxides such as (C₁₀-C₁₄)alkylamine oxides or N—(C₁₀-C₁₄)acylaminopropylmorpholine oxides; silicone surfactants; and mixtures thereof.

The nonionic surfactants may preferably be chosen from monooxyalkylenated, polyoxyalkylenated, monoglycerolated or polyglycerolated nonionic surfactants. The oxyalkylene units are more particularly oxyethylene or oxypropylene units, or a combination thereof, and are preferably oxyethylene units.

Examples of monooxyalkylenated or polyoxyalkylenated nonionic surfactants that may be mentioned include:
monooxyalkylenated or polyoxyalkylenated (C₅-C₂₄)alkylphenols,
saturated or unsaturated, linear or branched, monooxyalkylenated or polyoxyalkylenated C₈-C₃₀ alcohols,
saturated or unsaturated, linear or branched, monooxyalkylenated or polyoxyalkylenated C₈-C₃₀ amides,
esters of saturated or unsaturated, linear or branched, C₈-C₃M acids and of polyalkylene glycols,
monooxyalkylenated or polyoxyalkylenated esters of saturated or unsaturated, linear or branched, C₈-C₃₀ acids and of sorbitol,
saturated or unsaturated, monooxyalkylenated or polyoxyalkylenated plant oils, condensates of ethylene oxide and/or of propylene oxide, inter alia, alone or as mixtures.

The surfactants preferably contain a number of moles of ethylene oxide and/or of propylene oxide of between 1 and 100 and most preferably between 2 and 50. According to one of the embodiments of the present invention, the polyoxyalkylenated nonionic surfactants are chosen from polyoxyethylenated fatty alcohol (polyethylene glycol ether of fatty alcohol) and polyoxyethylenated fatty ester (polyethylene glycol ester of fatty acid).

Examples of polyoxyethylenated saturated fatty alcohol (or C₈-C₃₀ alcohols) that may be mentioned include the adducts of ethylene oxide with lauryl alcohol, especially those containing from 5 to 50 oxyethylene units and more particularly those containing from 7 to 12 oxyethylene units (Laureth-7 to Laureth-12, as the CTFA names); the adducts of ethylene oxide with behenyl alcohol, especially those containing from 9 to 50 oxyethylene units (Beheneth-9 to Beheneth-50, as the CTFA names); the adducts of ethylene oxide with cetearyl alcohol (mixture of cetyl alcohol and stearyl alcohol), especially those containing from 10 to 50 oxyethylene units (Ceteareth-10 to Ceteareth-50, for example, Ceteareth-33, as the CTFA names); the adducts of ethylene oxide with cetyl alcohol, especially those containing from 10 to 50 oxyethylene units (Ceteth-10 to Ceteth-50, as the CTFA names); the adducts of ethylene oxide with stearyl alcohol, especially those containing from 10 to 50 oxyethylene units (Steareth-10 to Steareth-50, for example, Steareth-20, as the CTFA names); the adducts of ethylene oxide with isostearyl alcohol, especially those containing from 10 to 50 oxyethylene units (Isosteareth-10 to Isosteareth-50, as the CTFA names); and mixtures thereof.

Examples of polyoxyethylenated unsaturated fatty alcohol (or C₈-C₃₀ alcohols) that may be mentioned include the adducts of ethylene oxide with oleyl alcohol, especially those containing from 2 to 50 oxyethylene units and more particularly those containing from 10 to 40 oxyethylene units (Oleth-10 to Oleth-40, as the CTFA names); and mixtures thereof.

As examples of monoglycerolated or polyglycerolated nonionic surfactants, monoglycerolated or polyglycerolated C₈-C₄₀ alcohols are preferably used.

In particular, the monoglycerolated or polyglycerolated C₈-C₄₀ alcohols correspond to the following formula:

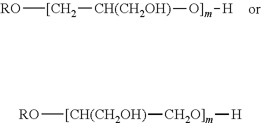

in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the present invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

Among the monoglycerolated or polyglycerolated alcohols, it is preferable to use the C₈/C₁₀ alcohol containing 1 mol of glycerol, the C₁₀/C₁₂ alcohol containing 1 mol of glycerol and the C₁₂ alcohol containing 1.5 mol of glycerol.

The monoglycerolated or polyglycerolated C₈-C₄₀ fatty esters may correspond to the following formula:

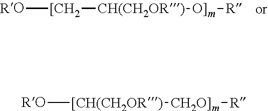

in which each of R′, R″ and R′″ independently represents a hydrogen atom, or a linear or branched C₈-C₄₀ and preferably C₈-C₃₀ alkyl-CO— or alkenyl-CO-radical, with the proviso that at least one of R′, R″ and R′″ is not a hydrogen atom, and m represents a number ranging from 1 to 30 and preferably from 1.5 to 10.

Examples of polyoxyethylenated fatty esters that may be mentioned include the adducts of ethylene oxide with esters of lauric acid, palmitic acid, stearic acid or behenic acid, and mixtures thereof, especially those containing from 9 to 100 oxyethylene units, such as PEG-9 to PEG-50 laurate (CTFA names: PEG-9 laurate to PEG-50 laurate); PEG-9 to PEG-50 palmitate (CTFA names: PEG-9 palmitate to PEG-50 palmitate); PEG-9 to PEG-50 stearate (CTFA names: PEG-9 stearate to PEG-50 stearate); PEG-9 to PEG-50 palmitostearate; PEG-9 to PEG-50 behenate (CTFA names: PEG-9 behenate to PEG-50 behenate); polyethylene glycol 100 EO monostearate (CTFA name: PEG-100 stearate); and mixtures thereof.

According to one of the embodiments of the present invention, the nonionic surfactant may be selected from esters of polyols with fatty acids with a saturated or unsaturated chain containing for example from 8 to 24 carbon atoms, preferably 12 to 22 carbon atoms, and polyoxyalkylenated derivatives thereof, preferably containing from 10 to 200, and more preferably from 10 to 100 oxyalkylene units, such as glyceryl esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sorbitol esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sugar (sucrose, maltose, glucose, fructose, and/or alkylglycose) esters of a C₅-C₂₄, preferably C₁₂-C₂₂, fatty acid or acids and polyoxyalkylenated derivatives thereof, preferably containing from 10 to 200, and more preferably from 10 to 100 oxyalkylene units; ethers of fatty alcohols; ethers of sugar and a C₅-C₂₄, preferably C₁₂-C₂₂, fatty alcohol or alcohols; and mixtures thereof.

As glyceryl esters of fatty acids, glyceryl stearate (glyceryl mono-, di- and/or tristearate) (CTFA name: glyceryl stearate), glyceryl laurate or glyceryl ricinoleate and mixtures thereof can be cited, and as polyoxyalkylenated derivatives thereof, mono-, di- or triester of fatty acids with a polyoxyalkylenated glycerol (mono-, di- or triester of fatty acids with a polyalkylene glycol ether of glycerol), preferably polyoxyethylenated glyceryl stearate (mono-, di- and/or tristearate), such as PEG-20 glyceryl stearate (mono-, di- and/or tristearate) can be cited.

Mixtures of these surfactants, for example, the product containing glyceryl stearate and PEG-100 stearate, marketed under the name ARLACEL 165 by Uniqema, and the product containing glyceryl stearate (glyceryl mono- and distearate) and potassium stearate marketed under the name TEGIN by Goldschmidt (CTFA name: glyceryl stearate SE), can also be used.

The sorbitol esters of C₅-C₂₄ fatty acids and polyoxyalkylenated derivatives thereof can be selected from sorbitan palmitate, sorbitan isostearate, sorbitan trioleate and esters of fatty acids and alkoxylated sorbitan containing for example from 20 to 100 EO, such as for example sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65, polyethylene sorbitan trioleate (polysorbate 85) or the compounds marketed under the trade names Tween 20 or Tween 60 by Uniqema.

As esters of fatty acids and glucose or alkylglucose, glucose palmitate, alkylglucose sesquistearates such as methylglucose sesquistearate, alkylglucose palmitates such as methylglucose or ethylglucose palmitate, methylglucoside fatty esters, the diester of methylglucoside and oleic acid (CTFA name: Methyl glucose dioleate), the mixed ester of methylglucoside and the mixture of oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, and mixtures thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

Mixtures of glycerides of alkoxylated plant oils such as mixtures of ethoxylated (200 EO) palm and copra (7 EO) glycerides can also be cited.

The nonionic surfactant according to the present invention preferably contains alkenyl or a branched C₁₂-C₂₂ acyl chain such as an oleyl or isostearyl group. More preferably, the nonionic surfactant according to the present invention is PEG-20 glyceryl triisostearate.

According to one of the embodiments of the present invention, the nonionic surfactant may be selected from copolymers of ethylene oxide and of propylene oxide, in particular copolymers of the following formula:

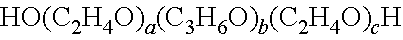

in which a, b and c are integers such that a+c ranges from 2 to 100, and b ranges from 14 to 60, and mixtures thereof.

According to one of the embodiments of the present invention, the nonionic surfactant may be selected from silicone surfactants. Non-limiting mention may be made of those disclosed in documents U.S. Pat. Nos. 5,364,633 and 5,411,744.

The silicone surfactant may preferably be a compound of formula (I):

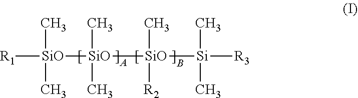

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; with the proviso that A and B are not simultaneously equal to zero;
x is an integer ranging from 1 to 6;
y is an integer ranging from 1 to 30; and
z is an integer ranging from 0 to 5.

According to one preferred embodiment of the present invention, in the compound of formula (I), the alkyl radical is a methyl radical, x is an integer ranging from 2 to 6 and y is an integer ranging from 4 to 30.

As examples of silicone surfactants of formula (I), mention may be made of the compounds of formula (II):

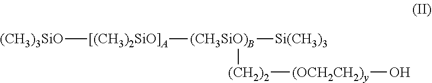

in which A is an integer ranging from 20 to 105, B is an integer ranging from 2 to 10 and y is an integer ranging from 10 to 20.

As examples of silicone surfactants of formula (I), mention may also be made of the compounds of formula (III):

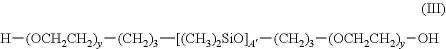

in which A′ and y are integers ranging from 10 to 20.

Compounds of the present invention which may be used are those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; A is 27, B is 3 and y is 12.

The compound Q4-3667 is a compound of formula (III) in which A is 15 and y is 13.

It is preferable that the (b) surfactant is selected from nonionic surfactants.

It is more preferable that the (b) surfactant is selected from polyglyceryl fatty acid esters.

The polyglyceryl fatty acid ester may have a polyglycerol moiety derived from 2 to 10 glycerols, preferably from 2 to 8 glycerols, and more preferably 2 to 6 glycerols. In other words, the polyglyceryl fatty acid ester may comprise from 2 to 10 polyglyceryl units, preferably 2 to 8 polyglyceryl units, and more preferably 2 to 6 polyglyceryl units. If all the polyglyceryl fatty acid ester has a shorter polyglyceryl chain (for example, less than 10 polyglyceryl units, preferably less than 8 polyglyceryl units, and more preferably less than 6 polyglyceryl units), the stability of the composition according to the present invention may be enhanced.

The polyglyceryl fatty acid ester may be chosen from the mono, di and tri esters of a linear or branched, saturated or unsaturated fatty acid, preferably saturated fatty acid, including from 4 to 32 carbon atoms, preferably from 8 to 26 carbon atoms, and more preferably from 10 to 20 carbon atoms, such as lauric acid, oleic acid, stearic acid, isostearic acid, capric acid, caprylic acid, and myristic acid.

It is preferable that the (b) surfactant be selected form polyglyceryl saturated or unsaturated fatty acid monoesters.

The polyglyceryl fatty acid ester may have an HLB (Hydrophilic Lipophilic Balance) value of from 4.0 to 16.0, preferably from 4.5 to 15.5, and more preferably from 5.0 to 15.0. The term HLB (“hydrophilic-lipophilic balance”) is well known to those skilled in the art, and reflects the ratio between the hydrophilic part and the lipophilic part in the molecule. If two or more polyglyceryl fatty acid esters are used, the HLB value is determined by the weighted average of the HLB values of all the polyglyceryl fatty acid esters.

The polyglyceryl fatty acid ester may be selected from the group consisting of PG-2 stearate (HLB: 5.0), PG-2 isostearate (HLB: 5.5), PG-2 oleate (HLB: 6.5), PG-2 caprate (HLB: 9.5), PG-2 laurate (HLB: 8.5), PG-4 oleate (HLB: 8.8), PG-4 laurate (HLB: 10.4), PG-4 isostearate (HLB: 8.2), PG-5 laurate (HLB: 15.8), PG-6 isostearate (HLB: 10.8), PG-3 cocoate (HLB: 12.0), PG-3 caprate (HLB: 10.0), PG-4 caprylate (HLB: 14), PG-4 caprate (HLB: 14.0), PG-5 myristate (HLB: 15.4), PG-5 stearate (HLB: 15.0), PG-5 oleate (HLB: 14.9), PG-6 caprylate (HLB: 14.6), PG-6 caprate (HLB: 13.1), PG-6 laurate (HLB: 14.5), and mixtures thereof.

It may be preferable that the (d) surfactant be selected from the group consisting of PG-4 caprate (HLB: 14.0), PG-2 isostearate (HLB: 5.5), and a mixture thereof.

It is preferable for the composition according to the present invention to comprise at least two polyglyceryl fatty acid esters.

It is preferable that the (d) surfactant be selected from
at least one first polyglyceryl fatty acid ester having an HLB value of 4.0 to 8.0, preferably 4.5 to 8.0, and more preferably 5.0 to 8.0;
at least one second polyglyceryl fatty acid ester having an HLB value of 12.0 to 16.0, preferably 12.0 to 15.5, and more preferably 12.0 to 15.0; and a mixture thereof.

The amount of the (d) surfactant(s) in the composition according to the present invention may be 0.001% by weight or more, preferably 0.01% by weight or more, and more preferably 0.1% by weight or more, relative to the total weight of the composition.

The amount of the (d) surfactant(s) in the composition according to the present invention may be 20% by weight or less, preferably 15% by weight or less, and more preferably 10% by weight or less, relative to the total weight of the composition.

The amount of the (d) surfactant(s) in the composition according to the present invention may be from 0.001% to 20% by weight, preferably from 0.01% to 15% by weight, and more preferably from 0.1% to 10% by weight, relative to the total weight of the composition.

[pH]

The pH of the composition according to the present invention may be from 2.0 to 9.0, preferably from 2.5 to 8.5, and more preferably from 3.0 to 8.0.

At a pH of from 2.0 to 9.0, the (a) polyion complex can be very stable.

The pH of the composition according to the present invention may be adjusted by adding at least one alkaline agent and/or at least one acid, other than the non-polymeric acid having two or more pKa values or salt(s) thereof or non-polymeric base having two or more pKb values or salt(s) thereof to be incorporated into the (a) polyion complex. The pH of the composition according to the present invention may also be adjusted by adding at least one buffering agent.

### (Alkaline Agent)

The composition according to the present invention may comprise at least one alkaline agent. Two or more alkaline agents may be used in combination. Thus, a single type of alkaline agent or a combination of different types of alkaline agents may be used.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and monohydrogenophosphates such as sodium phosphate or sodium monohydrogen phosphate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As an inorganic alkaline agent, sodium hydroxide is preferable.

The alkaline agent may be an organic alkaline agent. It is preferable that the organic alkaline agent be selected from the group consisting of monoamines and derivatives thereof; diamines and derivatives thereof; polyamines and derivatives thereof; basic amino acids and derivatives thereof; oligomers of basic amino acids and derivatives thereof; polymers of basic amino acids and derivatives thereof; urea and derivatives thereof; and guanidine and derivatives thereof.

As examples of the organic alkaline agents, mention may be made of alkanolamines such as mono-, di- and tri-ethanolamine, and isopropanolamine; urea, guanidine and their derivatives; basic amino acids such as lysine, ornithine or arginine; and diamines such as those described in the structure below:

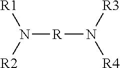

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof. Arginine, urea and monoethanolamine are preferable.

The alkaline agent(s) may be used in a total amount of from 0.01% to 15% by weight, preferably from 0.02% to 10% by weight, more preferably from 0.03% to 5% by weight, relative to the total weight of the composition, depending on their solubility.

### (Acid)

The composition according to the present invention may comprise at least one acid. Two or more acids may be used in combination. Thus, a single type of acid or a combination of different types of acids may be used.

As the acid, mention may be made of any inorganic or organic acids, preferably inorganic acids, which are commonly used in cosmetic products. A monovalent acid and/or a polyvalent acid may be used. A monovalent acid such as citric acid, lactic acid, sulfuric acid, phosphoric acid and hydrochloric acid (HCl) may be used. HCl is preferable.

The acid(s) may be used in a total amount of from 0.01% to 15% by weight, preferably from 0.02% to 10% by weight, more preferably from 0.03% to 5% by weight, relative to the total weight of the composition, depending on their solubility.

### (Buffering Agent)

The composition according to the present invention may comprise at least one buffering agent. Two or more buffering agents may be used in combination. Thus, a single type of buffering agent or a combination of different types of buffering agents may be used.

As the buffering agent, mention may be made of an acetate buffer (for example, acetic acid+sodium acetate), a phosphate buffer (for example, sodium dihydrogen phosphate+di-sodium hydrogen phosphate), a citrate buffer (for example, citric acid+sodium citrate), a borate buffer (for example, boric acid+sodium borate), a tartrate buffer (for example, tartaric acid+sodium tartrate dihydrate), Tris buffer (for example, tris(hydroxymethyl)aminomethane), and a Hepes buffer (4-(2-hydroxyethyl)-1-piperazineethanesulfonic acid).

### [Optional Additives]

The composition according to the present invention may comprise, in addition to the aforementioned components, components typically employed in cosmetics, specifically, oils such as ester oils and hydrocarbon oils, hydrophilic or lipophilic UV filters, hydrophilic or lipophilic thickeners, organic volatile or non-volatile solvents such as glycerin, glycols, and ethanol, silicones and silicone derivatives, natural extracts derived from animals or vegetables, waxes, and the like, within a range which does not impair the effects of the present invention,

The composition according to the present invention may comprise the above optional additive(s) in an amount of from 0.01% to 50% by weight, preferably from 0.05% to 30% by weight, and more preferably from 0.1% to 10% by weight, relative to the total weight of the composition.

### [Composition]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, preferably a skin care composition or a skin makeup composition, and more preferably a skin care composition.

The composition according to the present invention can be prepared by mixing the above essential and optional ingredients in accordance with any of the processes which are well known to those skilled in the art.

The composition according to the present invention can be prepared by simple or easy mixing with a conventional mixing means such as a stirrer. Thus, strong shearing by, for example, a homogenizer is not necessary. Also, heating is not necessary.

### [Film]

The composition according to the present invention can be used for easily preparing a film.

Thus, the present invention also relates to a process for preparing a film, preferably a cosmetic film, optionally with a thickness of preferably more than 0.1 μm, more preferably 0.5 μm or more, and even more preferably 1 μm or more, comprising:
applying onto a substrate, preferably a keratin substance, more preferably skin, the composition according to the present invention; and
drying the composition.

The upper limit of the thickness of the film according to the present invention is not limited. Thus, for example, the thickness of the film according to the present invention may be 1 mm or less, preferably 500 μm or less, more preferably 300 μm or less, and even more preferably 100 μm or less.

Since the process for preparing a film according to the present invention includes the steps of applying the composition according to the present invention onto a substrate, preferably a keratin substance, and more preferably skin, and of drying the composition, the process according to the present invention does not require any spin coating or spraying, and therefore, it is possible to easily prepare even a relatively thick film. Thus, the process for preparing a film according to present invention can prepare a relatively thick film without any special equipment such as spin coaters and spraying machines.

The film according to the present claims can be transparent or translucent, if the (b) hydrophilic antioxidant is used, and can provide antioxidative effects which can be long-lasting.

Even if the film according to the present invention is relatively thick, it is still thin and may be transparent, and therefore, may not be easy to perceive. Thus, the film according to the present invention can be used preferably as a cosmetic film.

The film according to the present claims can be less sticky, if the (b) lipophilic antioxidant is used, although it comprises a hyaluronic acid-based polymer, and can provide antioxidative effects which can be long-lasting.

If the substrate is not a keratin substance such as skin, the composition according to the present invention may be applied onto a substrate made from any material other than keratin.

The materials of the non-keratinous substrate are not limited. Two or more materials may be used in combination. Thus, a single type of material or a combination of different types of materials may be used. In any event, it is preferable that the substrate be flexible or elastic.

If the substrate is not a keratin substance, it is preferable that the substrate be water-soluble, because it is possible to leave the film according to the present invention by washing the substrate with water. As examples of the water-soluble materials, mention may be made of poly(meth) acrylic acids, polyethyleneglycols, polyacrylamides, polyvinylalcohol (PVA), starch, celluloseacetates, and the like. PVA is preferable.

If the non-keratinous substrate is in the form of a sheet, it may have a thickness of more than that of the film according to the present invention, in order to ease the handling of the film attached to the substrate sheet. The thickness of the non-keratinous substrate sheet is not limited, but may be from 1 μm to 5 mm, preferably from 10 μm to 1 mm, and more preferably from 50 to 500 μm.

It is more preferable that the film according to the present invention be releasable from the non-keratinous substrate. The mode of release is not limited. Therefore, the film according to the present invention may be peeled from the non-keratinous substrate, or released by the dissolution of the substrate sheet into a solvent such as water.

The present invention also relates to:
(1) A film, preferably a cosmetic film, optionally with a thickness of preferably more than 0.1 μm, more preferably 0.5 μm or more, and even more preferably 1 μm or more, prepared by a process comprising:
   applying onto a substrate, preferably a keratin substance, and more preferably skin, the composition according to the present invention; and
   drying the composition,
and
(2) A film, preferably a cosmetic film, optionally with a thickness of preferably more than 0.1 μm, more preferably 0.5 μm or more, and even more preferably 1 μm or more, comprising:
   (a) at least one polyion complex, comprising
   at least one cationic polymer and at least one anionic polymer,
   at least one cationic polymer and at least one amphoteric polymer,
   at least one anionic polymer and at least one amphoteric polymer, or
   at least one amphoteric polymer,
   at least one non-polymeric acid having two or more pKa values or salt(s) thereof, or
   at least one non-polymeric base having two or more pKb values or salt(s) thereof;
   (b) at least one hydrophilic or lipophilic antioxidant;
   and
   optionally (d) at least one surfactant, preferably selected from nonionic surfactants,
   and more preferably selected from polyglyceryl fatty acid esters,
   wherein
   the anionic polymer is selected from hyaluronic acid and salts thereof; and
   the amphoteric polymer is selected from cationized hyaluronic acid and salts thereof.

The above explanations regarding the cationic, anionic and amphoteric polymers as well as the above hydrophilic or lipophilic antioxidant and surfactant can apply to those in the above films (1) and (2).

The film thus obtained above can be self-standing. The term “self-standing” here means that the film can be in the form of a sheet and can be handled as an independent sheet without the assistance of a substrate or support. Thus, the term “self-standing” may have the same meaning as “self-supporting”.

It is preferable that the film according to the present invention be hydrophobic.

The term “hydrophobic” in the present specification means that the solubility of the polymer in water (preferably with a volume of 1 liter) at from 20 to 40° C., preferably from 25 to 40° C., and more preferably from 30 to 40° C. is less than 10% by weight, preferably less than 5% by weight, more preferably less than 1% by weight, and even more preferably less than 0.1% by weight, relative to the total weight of the polymer. It is most preferable that the polymer is not soluble in water.

If the film according to the present invention is hydrophobic, the film can have water-resistant properties, and therefore, it can remain on a keratin substance such as skin even if the surface of the keratin substance is wet due to, for example, sweat or rain. Thus, when the film according to the present invention provides any cosmetic effect, the cosmetic effect can last a long time.

On the other hand, the film according to the present invention can be easily removed from a keratin substance such as skin under alkaline conditions such as a pH of from 8 to 12, preferably from 9 to 11. Therefore, the film according to the present invention is difficult to remove with water, while it can be easily removed with a soap which can provide such alkaline conditions.

The film according to the present invention may comprise at least one biocompatible and/or biodegradable polymer layer. Two or more biocompatible and/or biodegradable polymers may be used in combination. Thus, a single type of biocompatible and/or biodegradable polymer or a combination of different types of biocompatible and/or biodegradable polymers may be used.

The term “biocompatible” polymer in the present specification means that the polymer does not have excess interaction between the polymer and cells in the living body including the skin, and the polymer is not recognized by the living body as a foreign material.

The term “biodegradable” polymer in the present specification means that the polymer can be degraded or decomposed in a living body due to, for example, the metabolism of the living body itself, or the metabolism of the microorganisms which may be present in the living body. Also, the biodegradable polymer can be degraded by hydrolysis.

If the film according to the present invention includes a biocompatible and/or biodegradable polymer, it is less irritable or not irritable to the skin, and does not cause any rash. In addition, due to the use of a biocompatible and/or biodegradable polymer, the cosmetic sheet according to the present invention can adhere well to the skin.

The film according to the present invention can be used for cosmetic treatments of keratin substances, preferably skin, in particular the face. The film according to the present invention can be in any shape or form. For example, it can be used as a full-face mask sheet, or a patch for a part of the face such as a cheek, nose, and around the eyes.

If the film according to the present invention includes at least one hydrophilic or water-soluble UV filter, it can provide UV shielding effects derived from the hydrophilic or water-soluble UV filter. Normally, a hydrophilic or water-soluble UV filter can be removed from the surface of a keratinous substrate such as skin by water such as sweat and rain. However, since the hydrophilic or water-soluble UV filter is included in the film according to the present invention, it is difficult for the hydrophilic or water-soluble UV filter to be removed by water, thereby resulting in long-lasting UV shielding effects.

### [Cosmetic Process and Use]

The present invention also relates to:
a cosmetic process for a keratin substance such as skin, comprising: applying to the keratin substance the composition according to the present invention; and drying the composition to form a cosmetic film on the keratin substance; and
a use of the composition according to the present invention for the preparation of a cosmetic film on a keratin substance such as skin.

The cosmetic process here means a non-therapeutic cosmetic method for caring for and/or making up the surface of a keratin substance such as skin.

In both the above process and use, the above cosmetic film is resistant to water with a pH of 7 or less, and is removable with water with a pH of more than 7, preferably 8 or more, and more preferably 9 or more.

In other words, the above cosmetic film can be water-resistant under neutral or acidic conditions such as a pH of 7 or less, preferably in a range of 6 or more and 7 or less, and more preferably in a range of 5 or more and 7 or less, while the above cosmetic film can be removed under alkaline conditions such as a pH of more than 7, preferably 8 or more, and more preferably 9 or more. The upper limit of the pH is preferably 13, more preferably 12, and even more preferably 11.

Accordingly, the above cosmetic film can be water-resistant, and therefore, it can remain on a keratin substance such as skin even if the surface of the keratin substance is wet due to, for example, sweat or rain. On the other hand, the above cosmetic film can be easily removed from a keratin substance such as skin under alkaline conditions. Therefore, the film according to the present invention is difficult to remove with water, while it can be easily removed with a soap which can provide alkaline conditions.

If the above cosmetic film includes a UV filter which may be present in the composition according to the present invention, the above cosmetic film can protect a keratin substance such as skin from UV rays, thereby limiting the darkening of the skin, improving the colour and uniformity of the complexion, and/or treating aging of the skin.

Furthermore, the above cosmetic film may have cosmetic effects such as capturing sebum, matting the appearance of a keratin substrate such as skin, absorbing or adsorbing malodour, and/or protecting the keratin substance from, for example, dirt or pollutants, due to the properties of the polyion complex in the cosmetic film, even if the cosmetic film does not include any cosmetic active ingredient.

In addition, the above cosmetic film may immediately change or modify the appearance of the skin by changing light reflection on the skin and the like, even if the cosmetic film does not include any cosmetic active ingredient. Therefore, it may be possible for the above cosmetic film to conceal skin defects such as pores or wrinkles. Further, the above cosmetic film may immediately change or modify the feel to the touch of the skin by changing the surface roughness on the skin and the like. Furthermore, the above cosmetic film may immediately protect the skin by covering the surface of the skin and shielding the skin, as a barrier, from environmental stresses such as pollutants, contaminants and the like.

The above cosmetic effects can be adjusted or controlled by changing the chemical composition, the thickness and/or the surface roughness of the above cosmetic film.

If the above cosmetic film includes at least one additional cosmetic active ingredient such as oil, the cosmetic film can have cosmetic effects provided by the additional cosmetic active ingredient(s). For example, if the cosmetic film includes at least one cosmetic active ingredient selected from anti-aging agents other than the (b) hydrophilic or lipophilic antioxidant, anti-sebum agents, deodorant agents, anti-perspirant agents, whitening agents and a mixture thereof, the cosmetic film can treat the aging of the skin, absorbing sebum on the skin, controlling odors on the skin, controlling perspiration on the skin, and/or whitening of the skin.

It is also possible to apply a makeup cosmetic composition onto the cosmetic film or sheet according to the present invention after it has been applied onto the skin.

### EXAMPLES

The present invention will be described in a more detailed manner by way of examples.

However, they should not be construed as limiting the scope of the present invention.

### Examples 1A-2A and Comparative Example 1A

### [Preparation]

Each of the compositions according to Examples 1A-2A and Comparative Example 1A was prepared by mixing the ingredients shown in Table 1A. The numerical values for the amounts of the ingredients in Table 1A are all based on “% by weight” as active materials.

TABLE 1A

| | | | Comp. |
|---|---|---|---|
| | Ex. 1A | Ex. 2A | Ex. 1A |
| Water | qsp 100 | qsp 100 | qsp 100 |
| Ascorbic Acid | 3.000 | 3.000 | 3.000 |
| Polyglyceryl-4 Caprate | — | 3.120 | — |
| Polyglyceryl-2 Isostearate | — | 0.050 | — |
| Phytic Acid | 0.138 | 0.138 | — |
| Polyepsilon-Lysine | 0.100 | 0.100 | — |
| Sodium Hydroxide | 0.032 | 0.032 | — |
| Sodium Hyaluronate (M.W. 20-50 kDa) | 1.000 | 1.000 | 1.000 |
| Sodium Hyaluronate (M.W. 1,000-1,400 kDa) | 0.500 | 0.500 | 0.500 |
| Preservative | q.s. | q.s. | q.s. |
| Transparency | Very Good | Very Good | Very Good |
| Lasting of Anti-Oxidative Effect | Good | Very Good | Poor |

### [Evaluations]

### (Transparency)

The appearance of each of the compositions according to Examples 1A-2A and Comparative Example 1A was evaluated. Specifically, each composition was taken into a transparent cylindrical glass container and evaluated in terms of the easiness of seeing a background through the container, and each composition was dropped onto a hand and evaluated in terms of the easiness of seeing the skin of the hand, in accordance with the following criteria:
Very Good: The background can be easily seen through the container (transparent)
Good: The background can be seen through the container, although slightly hazy (translucent)
Fair: The background cannot be seen through the container, while the skin can be easily seen (translucent)
Poor: The skin cannot be seen (opaque)

The results are shown in Table 1A.

### (Lasting of Anti-Oxidative Effect)

0.6 g of a β-carotene solution (solvent: caprylic/capric triglyceride) with a concentration of 0.05% by weight, and 0.2 g of each of the compositions according to Examples 1A-2A and Comparative Example 1A were mixed to obtain a mixture.

0.4 g of the above mixture was applied onto a filter (glass microfiber filter).

The filter was rinsed off with tap water (300 mL, 23-27° C.).

The above filter was exposed to UV rays with a wavelength of 365 nm for 30 minutes to oxidize β-carotene.

If β-carotene is oxidized, the original yellow color derived from β-carotene fades.

The b* values in the L*a*b* (CIE1976) space before and after the UV exposure (b* (T=0) and b* (T=30 min), respectively) of the filter were measured by a spectrocolorimeter (CM-700d made by KONICA MINOLTA), and the difference in the b* (T=0) value and the b* (T=30 min) value was determined as Ab*. It should be noted that the b* (T=0) value of the filter before being rinsed off was between 45 and 65.

The Δb* and the b* (T=30 min) values were evaluated in accordance with the following criteria.
Very Good: Δb*<10 and b* (T=30 min)>30
Good: Δb*<10 and 30>b* (T=30 min)>20
Poor: Δb*<10 and 20>b* (T=30 min)
Very Poor: Δb*>10

The results are shown in Table 1A.

### SUMMARY

The compositions according to Examples 1A and 2A can have transparent or translucent aspects.

It is clear from the comparison between Examples 1A-2A and Comparative Example 1 that the use of a polyion complex can lengthen anti-oxidative effects.

It is clear from the comparison between Example 1A and Example 2A that the use of a surfactant such as a polyglyceryl fatty acid ester can further lengthen anti-oxidative effects even under the conditions of rinsing off.

### Examples 1B-2B and Comparative Examples 1B-2B

### [Preparation]

Each of the compositions according to Examples 1B-2B and Comparative Examples 1B-2B was prepared by mixing the ingredients shown in Table 1B. The numerical values for the amounts of the ingredients in Table 1B are all based on “% by weight” as active materials.

TABLE 1B

| | | | Comp. | Comp. |
|---|---|---|---|---|
| | Ex. 1B | Ex. 2B | Ex. 1B | Ex. 2B |
| Water | qsp 100 | qsp 100 | qsp 100 | qsp 100 |
| Tocopherol | 0.10 | 0.10 | — | 0.10 |
| Polyglyceryl-4 Caprate | — | 3.12 | 3.12 | — |
| Polyglyceryl-2 Isostearate | — | 0.05 | 0.05 | — |
| Phytic Acid | 0.14 | 0.14 | 0.14 | — |
| Polyepsilon-Lysine | 0.10 | 0.10 | 0.10 | — |
| Sodium Hydroxide | 0.03 | 0.03 | 0.03 | — |
| Sodium Hyaluronate | 1.00 | 1.00 | 1.00 | 1.00 |
| (M.W. 20-50 kDa) | | | | |
| Sodium Hyaluronate | 0.50 | 0.50 | 0.50 | 0.50 |
| (M.W. 1,000-1,400 kDa) | | | | |
| Ethanol | 3.00 | 3.00 | 3.00 | 3.00 |
| Glycerin | 3.00 | 3.00 | 3.00 | 3.00 |
| Betaine | 1.00 | 1.00 | 1.00 | 1.00 |
| Ammonium Polyacryloyldimethyl | 0.40 | 0.40 | 0.40 | 0.40 |
| Taurate | | | | |
| Isopropyl Myristate | 0.32 | 0.32 | 0.32 | 0.32 |
| Isononyl Isononanoate | 0.32 | 0.32 | 0.32 | 0.32 |
| Trisodium Ethylenediamine | 0.25 | 0.25 | 0.25 | 0.25 |
| Disuccinate | | | | |
| Hydroxypropyl Methylcellulose | 0.10 | 0.10 | 0.10 | 0.10 |
| Preservative | q.s. | q.s. | q.s. | q.s. |
| Stickiness | Very Good | Good | Poor | Good |
| Lasting of Anti-Oxidative Effect | Good | Very Good | Very Poor | Poor |

### [Evaluations]

### (Stickiness)

Three panelists evaluated the texture, in terms of stickiness, of each of the compositions according to Examples 1B-2B and Comparative Examples 1B-2B at the timing of during and after application of the composition. Specifically, each panelist applied each composition on his or her hand and spread it to evaluate a moisturizing feeling, and graded it from 1 (low) to 5 (high). It was then classified in the following three categories based on the average of the grade:
Very Good: from 4 to 5 (Not Sticky)
Good: from 3 to less than 4 (Slightly Sticky)
Poor: more than 2 and less than 3 (Sticky)
Very Poor: from 1 to 2 (Very Sticky)

The results are shown in Table 1B.

### (Lasting of Anti-Oxidative Effect)

0.6 g of a β-carotene solution (solvent: caprylic/capric triglyceride) with a concentration of 0.05% by weight, and 0.2 g of each of the compositions according to Examples 1B-2B and Comparative Examples 1B-2B were mixed to obtain a mixture.

0.4 g of the above mixture was applied onto a filter (glass microfiber filter).

The filter was rinsed off with tap water (300 mL, 23-27° C.).

The above filter was exposed to UV rays with a wavelength of 365 nm for 30 minutes to oxidize β-carotene.

If β-carotene is oxidized, the original yellow color derived from β-carotene fades.

The b* values in the L*a*b* (CIE1976) space before and after the UV exposure (b* (T=0) and b* (T=30 min), respectively) of the filter were measured by a spectrocolorimeter (CM-700d made by KONICA MINOLTA), and the difference in the b* (T=0) value and the b* (T=30 min) value was determined as Δb*. It should be noted that the b* (T=0) value of the filter before being rinsed off was between 45 and 65.

The Δb* and the b* (T=30 min) values were evaluated in accordance with the following criteria.
Very Good: Δb*<10 and b* (T=30 min)>30
Good: Δb*<10 and 30>b* (T=30 min)>20
Poor: Δb*<10 and 20>b* (T=30 min)
Very Poor: Δb*>10

The results are shown in Table 1B.

### (Summary)

The compositions according to Examples 1B and 2B are good or very good in terms of stickiness and can provide good or very good long lasting antioxidative effects.

It is clear from the comparison between Example 1B and Comparative Example 2B that the use of a polyion complex can reduce stickiness and lengthen anti-oxidative effects.

It is clear from the comparison between Example 2B and Comparative Example 1B that the use of tocopherol can enhance not only antioxidative effects but also improve stickiness.

It is clear from the comparison between Example 1B and Example 2B that the use of a surfactant such as a polyglyceryl fatty acid ester can further lengthen anti-oxidative effects even under the conditions of rinsing off.

1. A composition, comprising:
(a) at least one polyion complex, comprising
at least one cationic polymer and at least one anionic polymer,
at least one cationic polymer and at least one amphoteric polymer,
at least one anionic polymer and at least one amphoteric polymer, or
at least one amphoteric polymer,
and
at least one non-polymeric acid having two or more pKa values or salt(s) thereof, or
at least one non-polymeric base having two or more pKb values or salt(s) thereof;
(b) at least one hydrophilic or lipophilic antioxidant; and
(c) water,
wherein
the anionic polymer is selected from hyaluronic acid, salts thereof, and derivatives thereof, and
the amphoteric polymer is selected from cationized hyaluronic acid and salts thereof.

2. The composition according to claim 1, wherein the cationized hyaluronic acid has at least one quaternary ammonium group-containing group and has a degree of cationization of 0.05 to 0.6, preferably from 0.1 to 0.5, and more preferably from 0.15 to 0.4.

3. The composition according to claim 1, wherein the cationic polymer has at least one positively chargeable and/or positively charged moiety selected from the group consisting of a primary, secondary or tertiary amino group, a quaternary ammonium group, a guanidine group, a biguanide group, an imidazole group, an imino group, and a pyridyl group.

4. The composition according to claim 1, wherein the cationic polymer is selected from the group consisting of cyclopolymers of alkyldiallylamine and cyclopolymers of dialkyldiallylammonium such as (co)polydiallyldialkyl ammonium chloride, (co)polyamines such as (co)polylysines, cationic (co)polyaminoacids such as collagen, cationic cellulose polymers, and salts thereof.

5. The composition according to claim 1, wherein the total amount of the cationic and/or anionic and/or amphoteric polymer(s) in the composition is from 0.01% to 15% by weight, preferably from 0.03% to 10% by weight, and more preferably from 0.05% to 5% by weight, relative to the total weight of the composition.

6. The composition according to claim 1, wherein the non-polymeric acid having two or more pKa values or salt(s) thereof is an organic acid or salt(s) thereof, preferably a hydrophilic or water-soluble organic acid or salt(s) thereof, and more preferably phytic acid or salts thereof.

7. The composition according to claim 1, wherein the amount of the non-polymeric acid having two or more pKa values or salt(s) thereof or non-polymeric base having two or more pKb values or salt(s) thereof in the composition is from 0.001% to 15% by weight, preferably from 0.005% to 10% by weight, and more preferably from 0.01% to 5% by weight, relative to the total weight of the composition.

8. The composition according to claim 1, wherein the (b) hydrophilic antioxidant is selected from ascorbic acid, derivatives thereof, salts thereof, and mixtures thereof.

9. The composition according to claim 1, wherein the amount of the (b) hydrophilic antioxidant in the composition is from 0.01% to 30% by weight, preferably from 0.05% to 25% by weight, and more preferably from 0.1% to 20% by weight, relative to the total weight of the composition.

10. The composition according to claim 1, wherein the (b) lipophilic antioxidant is selected from the group consisting of pentaerythrityl tetra-di-t-butyl hydroxyhydrocinnamate, nordihydroguaiaretic acid, propyl gallate, butylated hydroxytoluene, butylated hydroxyanisole, ascorbyl palmitate, tocopherol and mixtures thereof.

11. The composition according to claim 1, wherein the amount of the (b) lipophilic antioxidant in the composition is from 0.001% to 15% by weight, preferably from 0.005% to 10% by weight, and more preferably from 0.01% to 5% by weight, relative to the total weight of the composition.

12. A process for preparing a film, preferably a cosmetic film, comprising:
applying onto a substrate, preferably a keratin substance, the composition according to claim 1; and
drying the composition.

13. A film, preferably a cosmetic film, prepared by a process comprising:
applying onto a substrate, preferably a keratin substance, the composition according to claim 1; and
drying the composition.

14. A film, preferably a cosmetic film, comprising:
(a) at least one polyion complex comprising
at least one cationic polymer and at least one anionic polymer,
at least one cationic polymer and at least one amphoteric polymer,
at least one anionic polymer and at least one amphoteric polymer, or
at least one amphoteric polymer, and
at least one non-polymeric acid having two or more pKa values or salt(s) thereof, or
at least one non-polymeric base having two or more pKb values or salt(s) thereof;
(b) at least one hydrophilic or lipophilic antioxidant;
and
optionally (d) at least one surfactant, preferably selected from nonionic surfactants, and more preferably selected from polyglyceryl fatty acid esters,
wherein
the anionic polymer is selected from hyaluronic acid, salts thereof, and derivatives thereof; and
the amphoteric polymer is selected from cationized hyaluronic acid and salts thereof.

15. A cosmetic process for a keratin substance such as skin, comprising applying to the keratin substance the composition according to claim 1; and
drying the composition to form a cosmetic film on the keratin substance.
